(19) United States
(12) Patent Application Publication
(10) Pub. No.: US 2019/0209331 A1
(43) Pub. Date: Jul. 11, 2019

(54) SYSTEMS AND METHODS OF IMPLANTS TO RESTORE PATIENT SPECIFIC FUNCTON

(71) Applicant: THE GENERAL HOSPITAL CORPORATION, Boston, MA (US)

(72) Inventors: Kartik Mangudi Varadarajan, Acton, MA (US); Orhun K. Muratoglu, Cambridge, MA (US); Henrik Malchau, Boston, MA (US)

(21) Appl. No.: 16/099,114

(22) PCT Filed: May 9, 2017
(86) PCT No.: PCT/US17/31712

§ 371 Date: Nov. 5, 2018

Related U.S. Application Data
(60) Provisional application No. 62/334,372, filed on May 10, 2016.

(51) Int. Cl. A61F 2/30; A61F 2/40; A61F 2/38; A61F 2/42; A61L 27/48; A61F 2/36; G16H 20/40; G06F 16/51; G16H 30/20

(52) U.S. Cl. CPC A61F 2/30942; A61F 2002/30963; A61F 2/3859; A61F 2/3868; A61F 2/3804; A61F 2/4202; A61F 2/4261; A61L 27/48; A61F 2/3601; G16H 20/40; G06F 16/51; G16H 30/20; A61F 2002/30952; A61F 2002/30945; A61F 2002/30948; A61F 2/4059

(57) ABSTRACT

A method of manufacturing at least one component of a joint prosthesis, the method comprising creating a first database representing an anatomy of an articulating bone of a joint of a subject; accessing a second database representing a geometry of a generic prosthetic component being sized to fit around an outer articulating surface of the articulating bone of the joint of the subject; creating a third database representing kinematic data of the subject's joint; merging the databases within a three-dimensional image-based medium. The generic prosthetic component is attached to the outer articulating surface of the articulating bone of the joint creating a prosthetic articulating surface that is moved through the kinematic data of the subject through a bearing template thereby modifying the bearing template to create a kinematically appropriate bearing surface; and manufacturing the component of the joint prosthesis to have a geometry corresponding to the kinematically appropriate bearing surface.

### CROSS-REFERENCES TO RELATED APPLICATIONS

This application claims priority from U.S. Patent Application No. 62/334,372 filed May 10, 2016.

### STATEMENT REGARDING FEDERALLY SPONSORED RESEARCH

Not Applicable.

### BACKGROUND OF THE INVENTION

### 1. Field of the Invention

The present invention relates to design of implants, including a library of implants, and methods for designing or selecting the most appropriate implant for a given patient.

### 2. Description of the Related Art

Implants used for knee replacement surgery generally consist of one or more femoral components, and one or more tibial components. A tibial component in turn may be composed of tibial baseplate/s (also called tibial tray/s), and tibial bearing/s (also called tibial insert/s) affixed to the tibial baseplate/s. Total knee replacement (TKR) represents the largest market segment within orthopedics with over 1.2 million procedures performed annually, representing over $7.5 billion in sales (Ref. 1, 2). While TKR enjoys high rates of survivorship, >20% of patients continue to be dissatisfied with the surgery due to residual pain and functional limitation (Ref. 3, 4). In a study of patients under 55 years, only 66% of patients indicated their knees felt normal, and 31-54% reported difficulties performing activities such as climbing stairs, and getting in and out of a car or chair (Ref. 3). These limitations of modern TKR implants have been related to mismatch between individual patient's knee anatomy and geometry of standard “off-the-shelf” implants, which are not designed on a patient specific basis. Herein the terms “generic”, “off-the-shelf”, and “standard” are used interchangeably to refer to prosthetic components/implants, which are not designed specifically for individual patients. Further, herein the terms “custom” and “patient-specific” are used interchangeably to refer to prosthetic components/implants, which are either designed specifically for individual patients or selected specifically for individual patients from a library of designs.

Current off-the-shelf knee implant systems provide a limited range of femoral components varying in anteroposterior (AP) and/or mediolateral (ML) sizes, a limited range of tibial baseplates varying in AP/ML sizes, and a limited range of tibial bearings varying in AP/ML sizes. FIG. 1 shows an example of an off-the-shelf knee implant system with 10 femoral component sizes, 8 tibial baseplate sizes and 4 tibial bearing sizes. For a given patient, the femoral component and tibial baseplate are selected from these available fixed range of AP/ML sizes to best fit the patient's knee anatomy. The tibial bearing size to be used is automatically determined/dictated by the size compatibility chart based on the selected tibial baseplate and femoral component sizes (FIG. 1). A tibial bearing of given AP and ML size is generally also available in different proximal-distal (PD) thicknesses to provide surgeons with some options to balance the soft-tissue tension during surgery.

These off-the-shelf implant systems are limited in their ability to restore the patient's unique knee motion pattern (kinematics). Patients with similar tibial and femoral bone sizes can have substantially different knee kinematics. For example, consider knee 1 and knee 2 from two patients shown in FIG. 2. Both knees would receive the same femoral component, tibial baseplate, and tibial bearing due to similar native tibial and femoral AP and ML dimensions (size 6 femoral, size 7 tibial baseplate, size D tibial bearing from FIG. 1). However, the patients have substantially different native knee kinematics as depicted by different patterns of medial/lateral femoral condyle (MFC/LFC) motions as a function of knee flexion, in the two patients (FIG. 2). For a given femoral and tibial baseplate design, the articular geometry of the tibial bearing has a major influence on knee kinematics. Looking at FIG. 1, it can be seen that with a typical off-the-shelf knee implant system all patients with a given tibial size would receive the same tibial bearing, having the same articular geometry, irrespective of the patient's individual knee kinematics. Consider another example shown in FIG. 3. Knees 3 and 4 from two patients have different tibial and femoral sizes and therefore they would receive different sizes of tibial baseplate and femoral component (Knee 3=size 3 femoral, size 3 tibial baseplate; Knee 4=size 4 femoral, size 4 tibial baseplate from FIG. 1). However, both of these patients would still receive the same tibial bearing (Size B, FIG. 1), having the same articular geometry. Thus, there is a need for improved knee implant systems that are designed to accommodate and/or better restore the patient's own knee function/kinematics.

The limited range of implant sizes/shapes within an off-the-shelf implant system means that the implant may not precisely match the native anatomy of a given patient. To address mismatch between geometry of off-the-shelf implants and individual patient's knee anatomy, some manufacturers offer fully-custom knee implants, wherein the metal femoral component, metal tibial baseplate, and polyethylene tibial bearings are created to match the geometry of the native bones, based on magnetic resonance imaging/computed tomography (MRI/CT) scan of the patient's knee. However, the increased manufacturing cost and lead time associated particularly with designing and manufacturing the custom metallic femoral and tibial baseplate components, makes this approach cost-prohibitive. Thus, there remains need for improved and cost-effective designs and methods of designing knee implants to restore patient-specific knee function.

Thus, there remains need for improved and cost-effective designs and methods of designing TKR implants to restore patient-specific knee function.

### SUMMARY OF THE INVENTION

The present invention relates to implants to restore patient-specific function, specifically TKR implants to restore patient-specific knee function.

In some embodiments, a method of manufacturing at least one component of a joint prosthesis is provided. The method can comprise (a) creating a first database representing a two-dimensional or three-dimensional anatomy of an articulating bone of a joint of a subject; (b) accessing a second database representing a two-dimensional or three-dimensional geometry of a generic prosthetic component; the generic prosthetic component being sized to fit around an outer articulating surface of the articulating bone of the joint of the subject; (c) creating a third database representing kinematic data of the subject's joint; (d) merging the first database, the second database, and the third database within a three-dimensional image based medium wherein the generic prosthetic component is attached to the outer articulating surface of the articulating bone of the joint creating a prosthetic articulating surface, the prosthetic articulating surface being moved through the kinematic data of the subject through a bearing template thereby modifying the bearing template to create a kinematically appropriate bearing surface for the prosthetic articulating surface; and (e) manufacturing the at least one component of the joint prosthesis to have a geometry corresponding to the modified bearing template having a kinematically appropriate bearing surface.

In some embodiments, a method of kinematic analysis of acquired image data of a subject for determining geometry of at least one component of a joint prosthesis for the subject is provided. The method can comprise: (a) creating a first database representing a two-dimensional or three-dimensional anatomy of an articulating bone of the joint of a subject; (b) accessing a second database representing a two-dimensional or three-dimensional geometry of a generic prosthetic component; the generic prosthetic component being sized to fit around an outer articulating surface of the articulating bone of the joint of the subject; (c) creating a third database representing kinematic data of the subject; (d) merging the first database, the second database, and the third database into a three-dimensional image based medium wherein the generic prosthetic component is attached to the outer articulating surface of the articulating bone of the joint of the subject creating a prosthetic articulating surface, the prosthetic articulating surface being moved through the kinematic data of the subject through a bearing template thereby modifying the bearing template to create a kinematically appropriate bearing surface for the prosthetic articulating surface; and (e) determining a kinematically appropriate geometry of at least one component of a joint prosthesis based on the modified bearing template having a kinematically appropriate bearing surface.

In some embodiments, a joint prosthesis is provided. The joint prosthesis can comprise one or more generic prosthetic components that can be configured to be attached to a first articulating bone of a joint. The joint prosthesis can further comprise one or more patient-specific prosthetic components that can be configured to be attached to a second articulating bone of the joint. At least one of the one or more generic prosthetic components can articulate against at least one of one or more patient-specific prosthetic components.

In some embodiments, a method of manufacturing at least one component of a joint prosthesis is provided. The method can comprise: (a) creating a first database representing a two-dimensional or three-dimensional anatomy of an articulating bone of a joint of a subject; (b) accessing a second database representing a two-dimensional or three-dimensional geometry of a first generic prosthetic component; the first generic prosthetic component being sized to fit around an outer articulating surface of the articulating bone of the joint of the subject; (c) merging the first database and the second database within a three-dimensional image based medium wherein the first generic prosthetic component is attached to the outer articulating surface of the articulating bone of the joint creating a first prosthetic articulating surface; (d) creating a third database representing any surface contour deviation of the first prosthetic articulating surface from the outer articulating surface of the articulating bone; (e) accessing a fourth database representing a two-dimensional or three-dimensional geometry of a second generic prosthetic component for the joint of the subject, the second generic prosthetic component being opposite the first generic prosthetic component; (f) determining a surface geometry of a second prosthetic articulating surface based on a comparison of the third database and the fourth database; and (g) manufacturing a bearing surface of a custom prosthetic component to have a geometry corresponding to the second prosthetic articulating surface.

The proposed solution for restoring patient-specific knee function, involves use of off-the-shelf femoral and tibial baseplate components combined with custom (patient-specific) polyethylene (PE) tibial bearings. This TKR construct is also referred to herein as a “custom-bearing” TKR. For a given femoral component and tibial baseplate design, the articular geometry of the tibial bearing has a major influence on knee kinematics. Thus it is possible to obtain better restoration of patient-specific knee kinematics, by varying the design of the tibial bearing alone. In the foregoing sections, novel methods for designing patient-specific (custom) bearings are described, which account for one or more of the following: (a) patient's unique anatomy, (b) patient's unique knee kinematics, (c) differences between geometries of the off-the-shelf femoral/tibial baseplate components and patient's knee anatomy, (d) position of the off-the-shelf femoral/tibial baseplate components relative to the native bones, and (e) position of the off-the-shelf femoral component and off-the-shelf tibial baseplate relative to each other. The custom bearings of the present invention are different from the patient-specific bearings of fully-custom knee implants (prior-art). In a fully-custom knee implant, the articulating surfaces of both the femoral component and the tibial bearing are designed based on the patient's anatomy, derived from CT/MRI imaging data. In contrast, the tibial bearings of the current invention are designed to work with off-the-shelf femoral components, and as such they need to account for differences between geometries of the off-the-shelf femoral component and the native femur, and the specific 3D position in which the off-the-shelf femoral component and tibial baseplates are planned to be installed by the surgeon during the operation.

Custom bearings of the current invention can be machined for each patient at minimal cost using standard equipment, without need for patient-specific femoral/tibial baseplate components required in a fully-custom knee implants (prior-art). In another embodiment of the invention, as an alternative to machining tibial bearings for each individual patient, the most appropriate bearing geometry for a given patient can be selected from a library of tibial bearings. Therefore the proposed invention could provide improved knee function at a fraction of the cost of a fully customized implant.

These and other features, aspects, and advantages of the present invention will become better understood upon consideration of the following detailed description, drawings, and appended claims.

### BRIEF DESCRIPTION OF THE DRAWINGS

FIG. 1 is a femoral component, tibial baseplate, and tibial bearing size compatibility chart for a typical off-the-shelf TKR implant system.

FIG. 2 is kinematics of two knees (Knee 1, Knee 2) from two different patients, represented as motion of medial femoral condyle center and lateral femoral condyle center (MFC, LFC) relative to tibia, as function of knee flexion angle.

FIG. 3 is kinematics of two knees (Knee 3, Knee 4) from two different patients, represented as motion of medial femoral condyle center and lateral femoral condyle center (MFC, LFC) relative to tibia, as function of knee flexion angle.

FIG. 4A is one embodiment of a workflow/methodology utilizing a kinematic approach for creating patient-specific tibial bearings starting with three-dimensional (3D) knee models based on CT/MRI.

FIG. 4B is one embodiment of a workflow/methodology utilizing a kinematic approach for creating patient-specific tibial bearings starting with two-dimensional (2D) radiographs.

FIG. 5 is one embodiment of a virtual carving process to generate patient-specific bearing articular geometry.

FIG. 6 is an off-the-shelf femoral and a tibial baseplate component mounted on 3D models of two patients. Sectional views a-a and b-b show sagittal plane view of cross-section taken through lateral condyle of femoral component and native femur of patients A and B respectively.

FIG. 7 is one embodiment of patient-specific bearings created for patients A and B of FIG. 6 through process described in FIG. 4.

FIG. 8A is one embodiment of medial/lateral cross-sections of patient-specific and off-the-shelf (non-patient specific) tibial bearing.

FIG. 8B is one embodiment of simulated kinematics for a knee with bearing designs of FIG. 8A showing motion of medial/lateral femoral condyle relative to tibia during a deep knee bend activity.

FIG. 9 is one embodiment of a virtual carving process to generate patient-specific articular surface and tibial post geometries of a patient-specific PS tibial bearing

FIG. 10 is one embodiment of simulated kinematics for a knee with patient-specific and off-the-shelf (non-patient specific) PS tibial bearing designs, both articulating with identical off-the-shelf femoral component and mating with identical off-the-shelf tibial baseplate.

FIG. 11A is one embodiment of a workflow/methodology utilizing a geometric approach for creating patient-specific tibial bearings starting with three-dimensional (3D) knee models based on CT/MRI.

FIG. 11B is one embodiment of a workflow/methodology utilizing a geometric approach for creating patient-specific tibial bearings starting with two-dimensional (2D) radiographs.

FIG. 12 is one embodiment of an off-the-shelf femoral and a tibial baseplate component mounted on 3D models of two patients. The bottom row shows transverse plane view of custom tibial bearings for patient A and patient B with different orientations of tibial articular surface low-point pathway, designed to accommodate differences in relative transverse plane rotation between femoral component and tibial baseplate in the two patients.

FIG. 13 is one embodiment of an off-the-shelf femoral and a tibial baseplate component mounted on 3D models of two patients. The bottom row shows transverse plane view of custom tibial bearings for patient A and patient B with different medial-lateral location of tibial articular surface low-point pathway, designed to accommodate differences in relative medial-lateral location of the femoral component relative to the tibial baseplate in the two patients.

FIG. 14A is one embodiment of a sagittal plane cross-section through an off-the-shelf femoral component mounted on the native femur of a patient (only the articular surface of the component and native femur shown), showing differences between component and native femur in regions 1 and 5.

FIG. 14B is one embodiment of a custom tibial bearing designed to account for differences in the articular geometry of femoral component and native femur.

FIG. 14C is one embodiment of a custom tibial bearing designed to account for differences in the articular geometry of femoral component and native femur.

FIG. 15A is one embodiment of a sagittal plane cross-section through an off-the-shelf femoral component mounted on the native femur of a patient (only the articular surface of the component and native femur shown), showing differences between component and native femur in regions 1 and 5.

FIG. 15B is one embodiment of a custom tibial bearing designed to account for differences in the articular geometry of femoral component and native femur.

FIG. 15C is one embodiment of a custom tibial bearing designed to account for differences in the articular geometry of femoral component and native femur.

FIG. 16A is one embodiment of a sagittal plane cross-section through an off-the-shelf femoral component mounted on the native femur of a patient (only the articular surface of the component and native femur shown), showing differences between component and native femur in regions 1, 3 and 5.

FIG. 16B is one embodiment of a custom tibial bearing designed to account for differences in the articular geometry of femoral component and native femur.

FIG. 16C is one embodiment of a custom tibial bearing designed to account for differences in the articular geometry of femoral component and native femur.

FIG. 17A is one embodiment of a sagittal plane cross-section through an off-the-shelf femoral component mounted on the native femur of a patient (only the articular surface of the component and native femur shown), showing differences between component and native femur in regions 1, 3 and 5.

FIG. 17B is one embodiment of a custom tibial bearing designed to account for differences in the articular geometry of femoral component and native femur.

FIG. 17C is one embodiment of a custom tibial bearing designed to account for differences in the articular geometry of femoral component and native femur.

FIG. 18A is one embodiment of a sagittal plane cross-section through an off-the-shelf femoral component mounted on the native femur of a patient (only the articular surface of the component and native femur shown), showing differences between component and native femur in regions 1 and 3.

FIG. 18B is one embodiment of a custom tibial bearing designed to account for differences in the articular geometry of femoral component and native femur.

FIG. 18C is one embodiment of a custom tibial bearing designed to account for differences in the articular geometry of femoral component and native femur.

FIG. 19A is one embodiment of a sagittal plane cross-section through an off-the-shelf femoral component mounted on the native femur of a patient (only the articular surface of the component and native femur shown), showing differences between component and native femur in regions 1 and 3.

FIG. 19B is one embodiment of a custom tibial bearing designed to account for differences in the articular geometry of femoral component and native femur.

FIG. 19C is one embodiment of a custom tibial bearing designed to account for differences in the articular geometry of femoral component and native femur.

FIG. 20A is one embodiment of a sagittal plane cross-section through an off-the-shelf femoral component mounted on the native femur of a patient (only the articular surface of the component and native femur shown), showing differences between component and native femur in regions 1, 3 and 5.

FIG. 20B is one embodiment of a custom tibial bearing designed to account for differences in the articular geometry of femoral component and native femur.

FIG. 20C is one embodiment of a custom tibial bearing designed to account for differences in the articular geometry of femoral component and native femur.

FIG. 21A is one embodiment of a sagittal plane cross-section through an off-the-shelf femoral component mounted on the native femur of a patient (only the articular surface of the component and native femur shown), showing differences between component and native femur in regions 1, 3 and 5.

FIG. 21B is one embodiment of a custom tibial bearing designed to account for differences in the articular geometry of femoral component and native femur.

FIG. 21C is one embodiment of a custom tibial bearing designed to account for differences in the articular geometry of femoral component and native femur.

FIG. 22 is one embodiment showing tibiofemoral kinematics to describe motion of the medial and lateral femoral condyle centers in three-dimensional space relative to a tibial coordinate system.

FIG. 23 is one embodiment of a library of bearings is designed to provide different tibiofemoral kinematic patterns, from which the most appropriate bearing can be selected for each patient.

FIG. 24 is one embodiment of a library of bearings is designed to provide different tibiofemoral kinematic patterns, from which the most appropriate bearing can be selected for each patient as compared to FIG. 1.

FIG. 25 is one embodiment of the medial and lateral tibial low-point pathway/s (LP/s) that can be varied to provide different kinematic patterns.

FIG. 26A is one embodiment of the medial and lateral tibial low-point pathway/s (LP/s) that can be varied to provide different kinematic patterns.

FIG. 26B is one embodiment that shows the lateral LP is parallel to the AP axis in a transverse plane, while the medial LP is angled.

FIG. 26C is one embodiment that shows anterior portions of medial and lateral LPs are angled towards the medial side of the knee relative to a bearing AP axis, while the posterior portion of medial and lateral LPs are parallel to the said AP axis.

FIG. 26D is one embodiment that shows an anterior portion of the lateral LP is angled towards the medial side of the knee relative to a bearing AP axis, while the posterior portion of medial and lateral LPs are parallel to the said AP axis.

FIG. 26E is one embodiment that shows the anterior and posterior portions of medial and lateral LPs are curved while the central portions are straight and parallel to a bearing AP axis.

FIG. 27A is one embodiment that shows the anterior portions of medial and lateral LPs are angled laterally relative to a bearing AP axis while the posterior portions are parallel to the said bearing AP axis.

FIG. 27B is one embodiment that shows the anterior portions of medial and lateral LPs are curved in a transverse plane with the concave side of the curve facing laterally, and the posterior portions of medial and lateral LPs are curved with the concave side of the curve facing medially.

FIG. 28A is one embodiment that shows both medial and lateral LPs are curved with the concave side of the curvature facing medially.

FIG. 28B is one embodiment that shows both medial and lateral LPs are curved with the concave side of the curvature facing medially, and both having same center of curvature.

FIG. 28C is one embodiment that shows both medial and lateral LPs are curved with the concave side of the curvature facing medially, and both having different centers of curvature.

FIG. 29A is one embodiment that shows both medial and lateral LPs are curved with the concave side of the curvature facing laterally, and both having different centers of curvature located outside the tibial bearing.

FIG. 29B is one embodiment that shows both medial and lateral LPs are curved with the concave side of the curvature facing laterally, and both having the same center of curvature.

FIGS. 30A to 30F show many embodiments of different bearings of the same size from the library having medial and lateral LPs lying along straight lines in a transverse plane, but with the LPs angled by different amounts and/or in different directions amongst the different bearings relative to a bearing AP axis.

FIG. 31A is one embodiment that shows the medial and lateral LPs lie along straight lines parallel to a bearing axis.

FIG. 31B is one embodiment that shows the anterior medial and lateral LPs are angled relative to the bearing axis.

FIG. 31C is one embodiment that shows the anterior medial and lateral LPs are angled by greater amount relative to the bearing axis than that in bearing of FIG. 31B.

FIG. 31D is one embodiment that shows the anterior medial and lateral LPs are angled towards the medial side relative to the bearing axis while the posterior medial and lateral LPs are angled towards the lateral side relative to the bearing axis.

FIG. 32A is one embodiment that shows the medial/lateral bearing articular profile is composed of 2 concave arcs of different radii.

FIG. 32B is one embodiment that shows the medial/lateral tibial articular profile is composed of an anterior concave arc, a central flat section (line segment) angled relative to the tibial bearing base, and a posterior arc

FIG. 32C is one embodiment that shows the medial/lateral tibial articular profile may be composed of an anterior concave arc, and a posterior flat section (line segment) angled relative to the tibial bearing base.

FIG. 33A is one embodiment that shows the medial/lateral tibial articular profile is composed of an anterior concave arc, a central convex arc, and a posterior concave arc.

FIG. 33B is one embodiment that shows the medial or lateral tibial articular profile is composed of an anterior concave arc, and a posterior concave arc.

FIG. 33C is one embodiment that shows the medial or lateral tibial articular profile may be composed of a convex arc.

FIGS. 34A to 34C are many embodiments that show bearings of given AP/ML size and PD thickness within the library may be configured to have different medial/lateral articular geometry by changing the slope of a given articular profile relative to the bearing base.

FIG. 35A is one embodiment that shows bearings of given size and thickness within the library may be configured to have different medial/lateral articular geometry by shifting a given articular profile in anterior or posterior direction.

FIG. 35B is one embodiment that shows bearings of given size and thickness within the library may be configured to have different medial/lateral articular geometry by shifting the articular low point in anterior or posterior direction

FIG. 35C is one embodiment that shows bearings of given size and thickness within the library may be configured to have different medial/lateral articular geometry by shifting the location of intersection of anterior and posterior arcs in anterior or posterior direction.

FIGS. 36A to 36B and FIGS. 37A to 37B are many embodiments that show bearings of given size and thickness within the library may be configured to have different anterior/posterior, and/or medial/lateral location of the tibial post.

FIGS. 38A to 38B are embodiments that show bearings of given size and thickness within the library may be configured to have tibial posts of different anteroposterior width.

FIGS. 39A to 39B are embodiments that show bearings of given size and thickness within the library may be configured to have tibial posts of different mediolateral width.

FIGS. 40A to 40B are embodiments that show bearings of given size and thickness within the library may be configured to have different orientations of the tibial post relative to each other, such as different internal and or external rotation in a transverse view.

FIGS. 41A to 41B are embodiments that show bearings of given size and thickness within the library may be configured to have different shapes of the anterior, posterior, medial, lateral or proximal surface/s of the tibial post.

FIG. 42 is one embodiment that shows patient-specific bearings for other joint replacement implants, including shoulder replacement implants (total shoulder, reverse shoulder etc.), ankle replacement implants, hip replacement implants, wrist replacement implants, elbow replacement implants, etc.

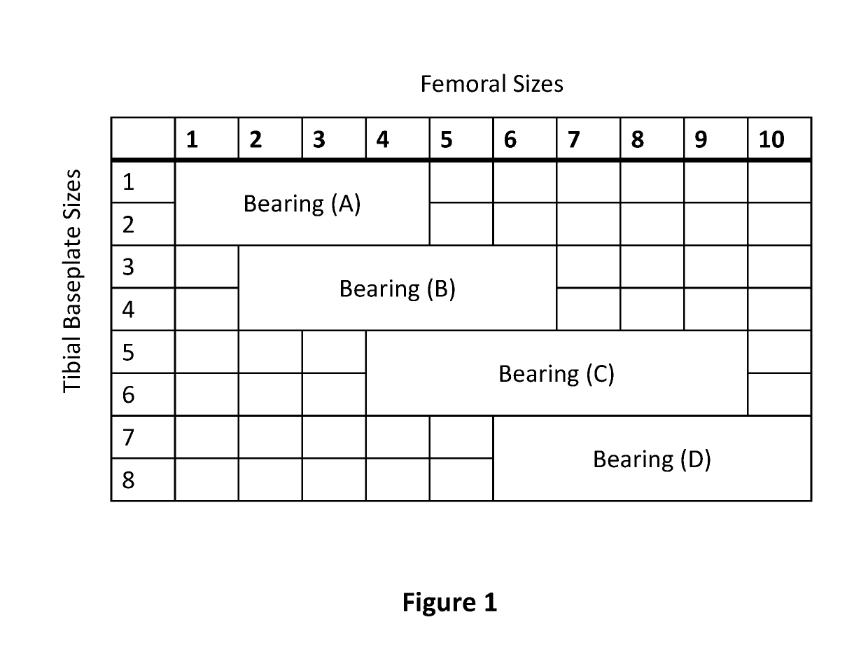

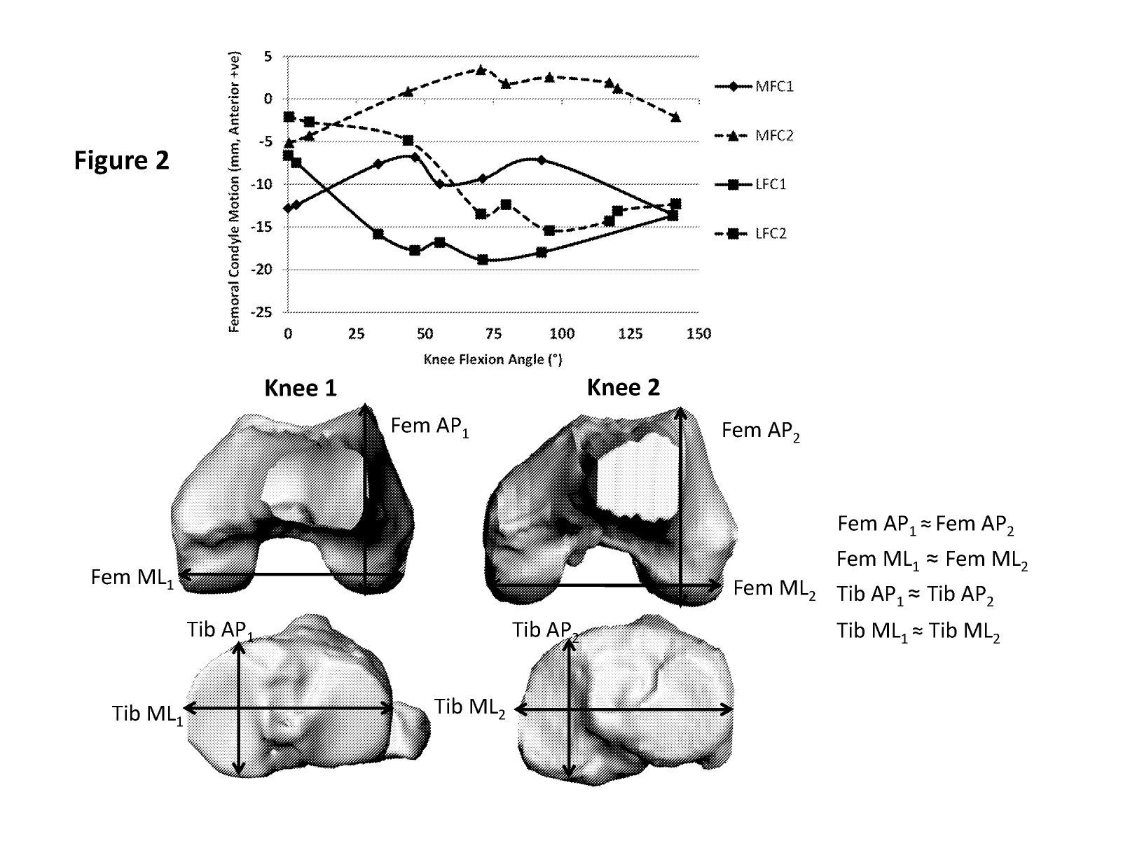

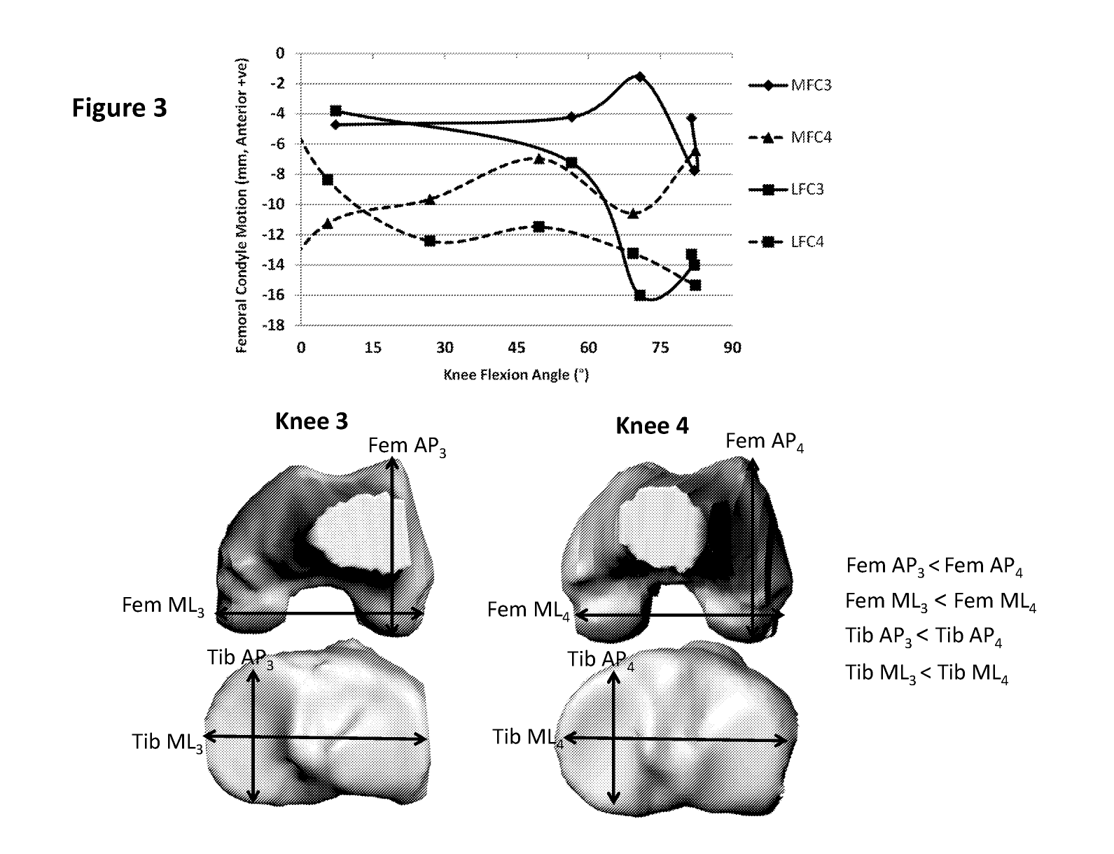

Like reference numerals will be used to refer to like parts from Figure to Figure in the following description of the drawings.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined by the claims. The features illustrated or described in connection with one exemplary embodiment may be combined with the features of other embodiments. Such modifications and variations are intended to be included within the scope of the present invention.

### Custom-Bearing Knee Implants

### A: Kinematic Approach to Designing Custom-Bearing Knee Implants

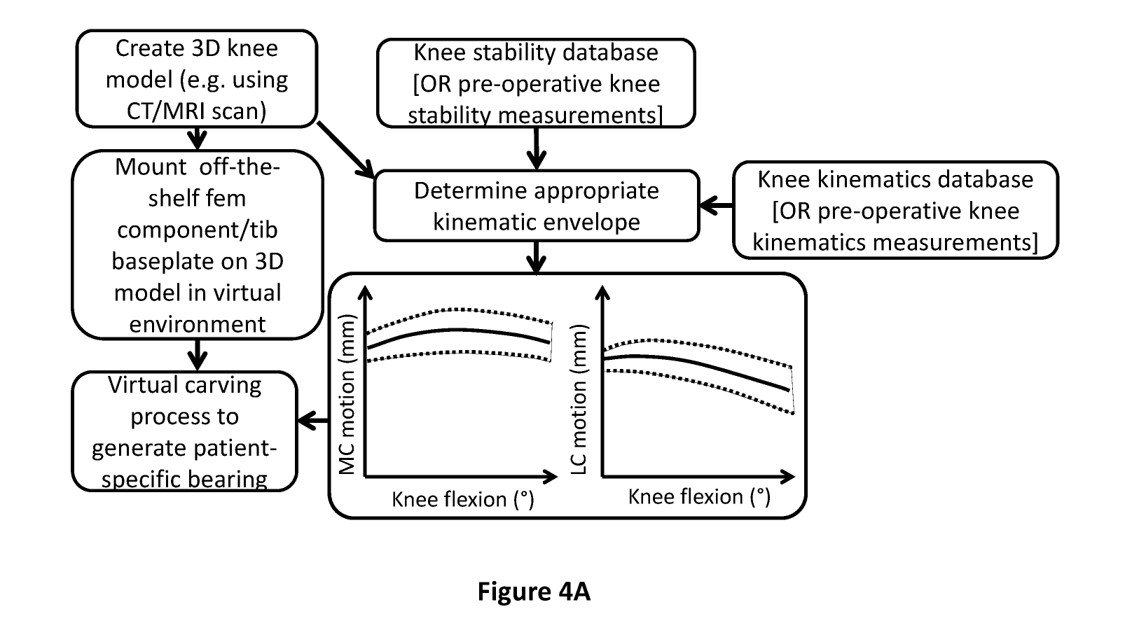

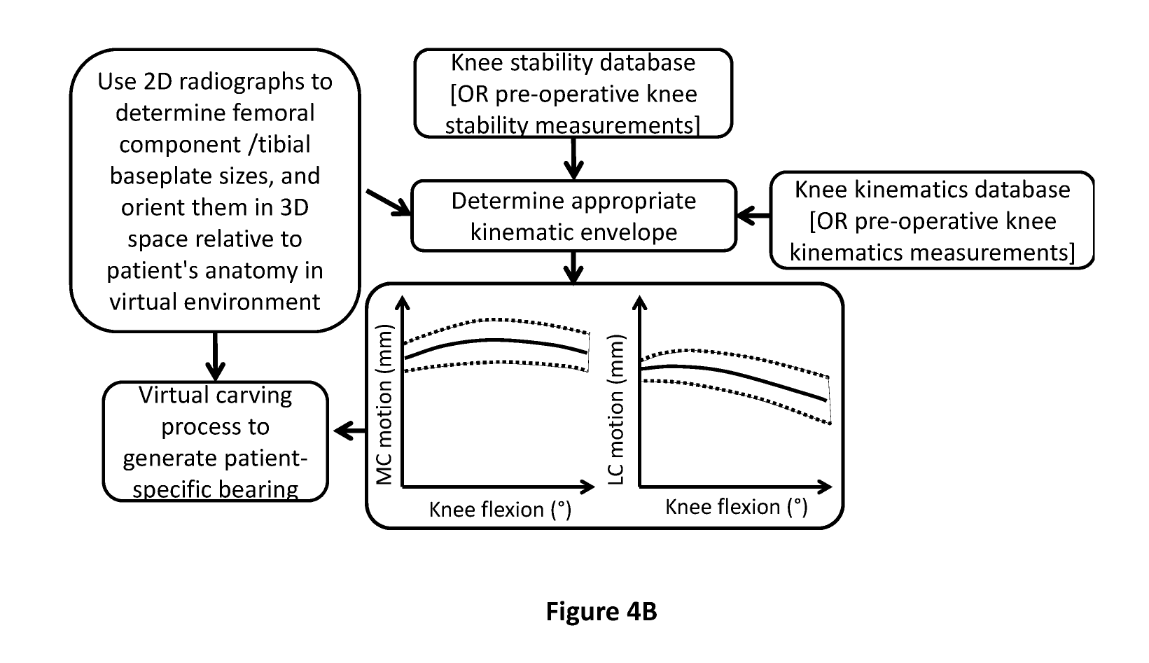

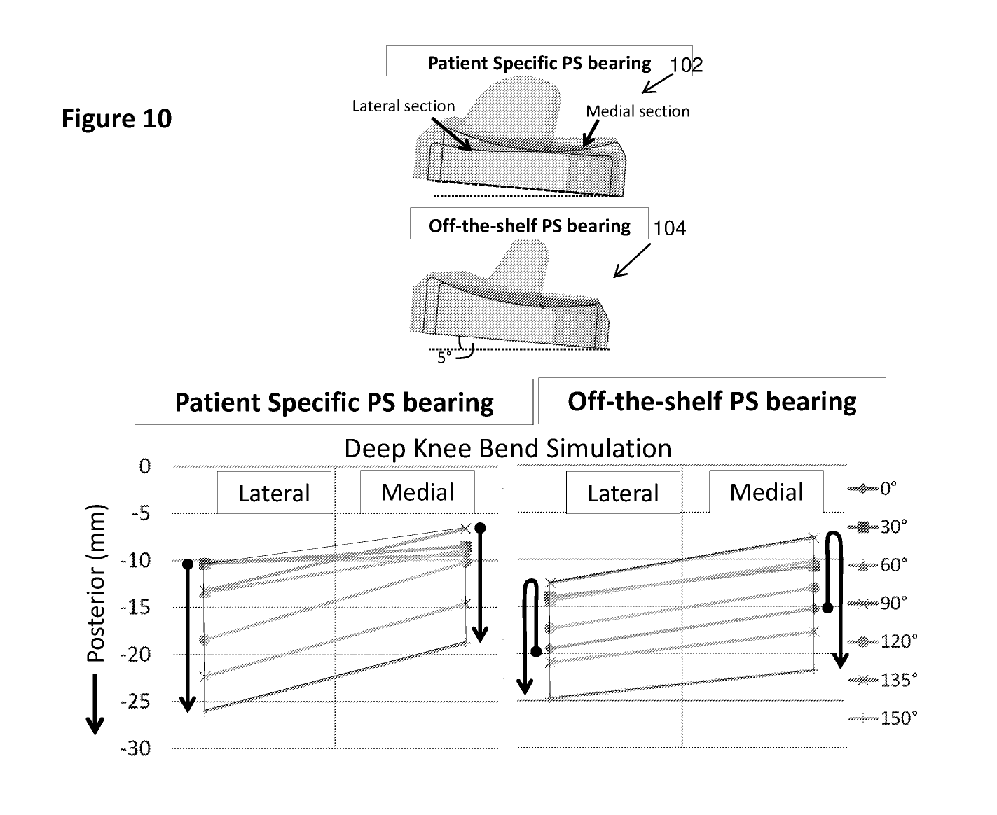

In one approach to designing custom/patient-specific tibial bearings, a 3D model of the patient's knee is created pre-operatively using CT/MRI, or other imaging modalities (FIG. 4A). Appropriately sized off-the-shelf femoral and tibial baseplate components are then mounted on the 3D knee model in a virtual three-dimensional image based environment (such as in a computer) in consultation with the surgeon. Alternatively, appropriately sized femoral and tibial baseplate components can be oriented in 3D space based on single-plane or multi-planar radiographic images of the patient's knee (Ref. 5, 6) (FIG. 4B). A computer algorithm is then used to move the femoral component relative to the tibial baseplate through a kinematic path/kinematic envelope appropriate for that patient. Simultaneously, the computer program carves out the corresponding articular surface of the tibial bearing from a tibial bearing template through a series of Boolean subtraction operations (FIG. 5). Various post-processing steps can then be carried out to finalize the tibial bearing geometry, including smoothening the geometry obtained from the Boolean operation, trimming the outer perimeter of the bearing according to the shape of the tibial baseplate in a transverse plane (FIG. 12), adding appropriate locking features on the back-side of the bearing (side opposite to articular surface) to mate with the tibial baseplate etc. This overall approach is referred to herein as a kinematic approach to designing custom bearing knee implants.

The kinematics of an individual knee are determined by activity specific muscle activation, stability provided by passive soft-tissues (e.g. ligaments, menisci), and bony anatomy of the knee. Therefore, kinematic envelope appropriate for designing custom tibial bearings for a given patient may be determined by using a combination of activity specific kinematics data, knee joint stability/laxity data, and anatomy data (FIGS. 4A, B). For example, first average knee kinematics obtained from a pre-existing kinematics database can be scaled/modified to account for patient-specific anthropometric measurements (e.g. bony dimensions, height, weight) to obtain a patient-specific kinematics profile. Alternatively, a patient-specific kinematics profile can be determined by recording the pre-operative kinematics of the patient's knee while they perform one or more activities such as deep knee bending, walking, stair climbing etc. using techniques such as single/bi-planar video fluoroscopy, marker based optical trackers, etc. Next, average native knee stability data obtained from a pre-existing database can be scaled/modified to account for patient-specific anthropometric measurements (e.g. bone dimensions from CT/MRI, height, weight) to obtain a patient-specific knee stability profile. Alternatively, pre-operative joint stability measurements (e.g. anterior/posterior drawer tests, internal/external rotation tests, varus/valgus distraction tests) performed on the patient's knee may be used to obtain a patient-specific knee stability profile. The patient-specific kinematic path/kinematic envelope to be used for creating the custom bearing, can then be obtained by combining the patient-specific kinematics profile (solid lines in graphs of FIGS. 4A, B) and the patient-specific stability profile (distance between dotted lines at given flexion angle, FIGS. 4A, B).

In some cases, there may be excessive changes in anatomy, kinematics or stability of the knee due to the advanced diseased state of the joint. In such cases it may not be appropriate to directly use the anatomy, kinematics or stability measurements of the diseased knee for creating the patient-specific bearing. In these situations, data of the contralateral knee of the patient can be used (after making appropriate adjustments such as mirroring of the data).

The patient-specific tibial bearing can be either machined from the carved surface following post-processing, or the closest bearing geometry can be selected from a pre-established library of bearings. Prior to surgery the surgeon would receive the patient-specific tibial bearing to be used with the off-the-shelf femoral/tibial baseplate components. The bearing can be assembled to/mated with the tibial baseplate via the use of one or more locking mechanisms similar to those used in various knee implants. For optimal outcome, the surgeon may also use tools such as computer navigation, custom guides, or haptic robots etc. to accurately reproduce the pre-operative plan regarding placement of the femoral and tibial baseplate components on the native bones.

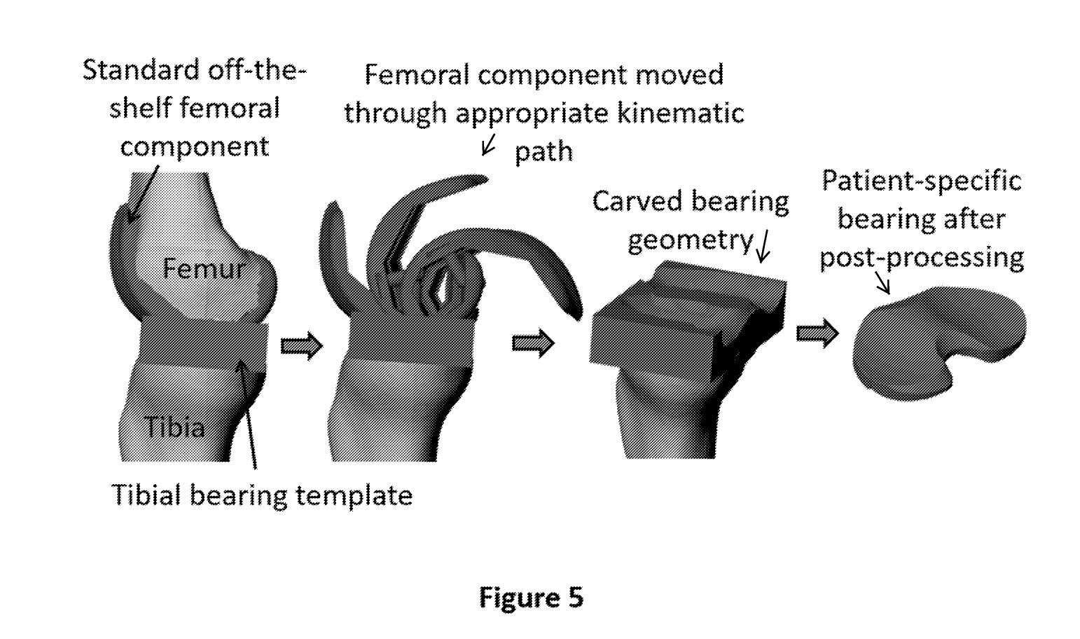

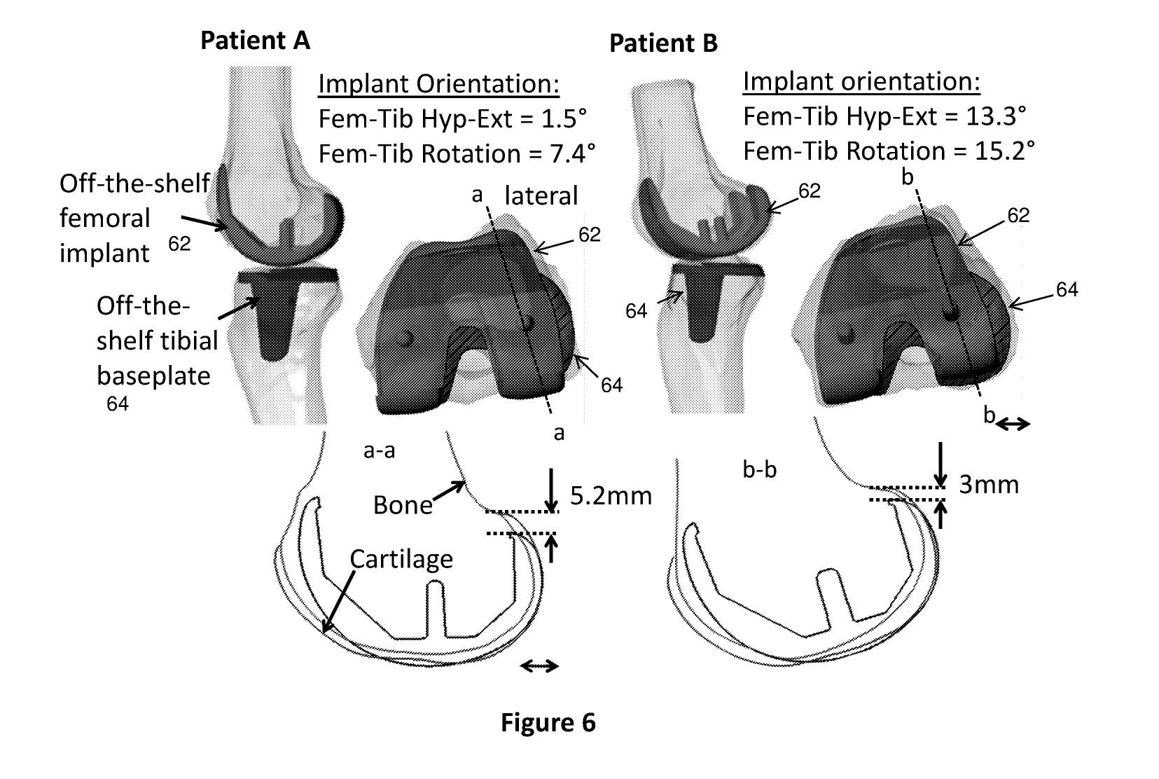

In FIG. 6, appropriate sized off-the-shelf femoral 62 and tibial baseplate 64 components are shown mounted on 3D knee models of 2 patients. Component orientations differ significantly between the two patients. In patient B, the femoral component is more hyper-extended and externally rotated relative to the tibial baseplate, than in subject 1 (˜12° and 8° more). Additionally, in patient B the femoral component covers the lateral femoral articular surface to a greater extent than in patient A (˜2.2 millimeters (mm) greater). These differences directly affect the geometry of the patient-specific tibial bearings 72 generated through the above described process/processes (FIG. 7). The lateral compartment of the custom bearing for patient B has a lower anterior lip on the lateral side, to accommodate the greater hyper-extension and external femoral rotation in this patient. Similarly, lateral compartment of the custom bearing for patient B has a lower posterior lip since the femoral component replaces the native femoral articular cartilage to a greater extent in this subject. This illustrates the rationale for utilizing custom bearings to accommodate patient-specific differences in position and/or fit of off-the-shelf implant components relative to the native anatomy. The geometry of the custom bearing obtained through the above described process/processes is a function of the patient-specific kinematic path/kinematic envelope, how the femoral component fits the native anatomy (e.g. initial femoral component extension relative to native femur), relative location/orientation of femoral component relative to tibial baseplate, and location/orientation of tibial baseplate relative to native tibia.

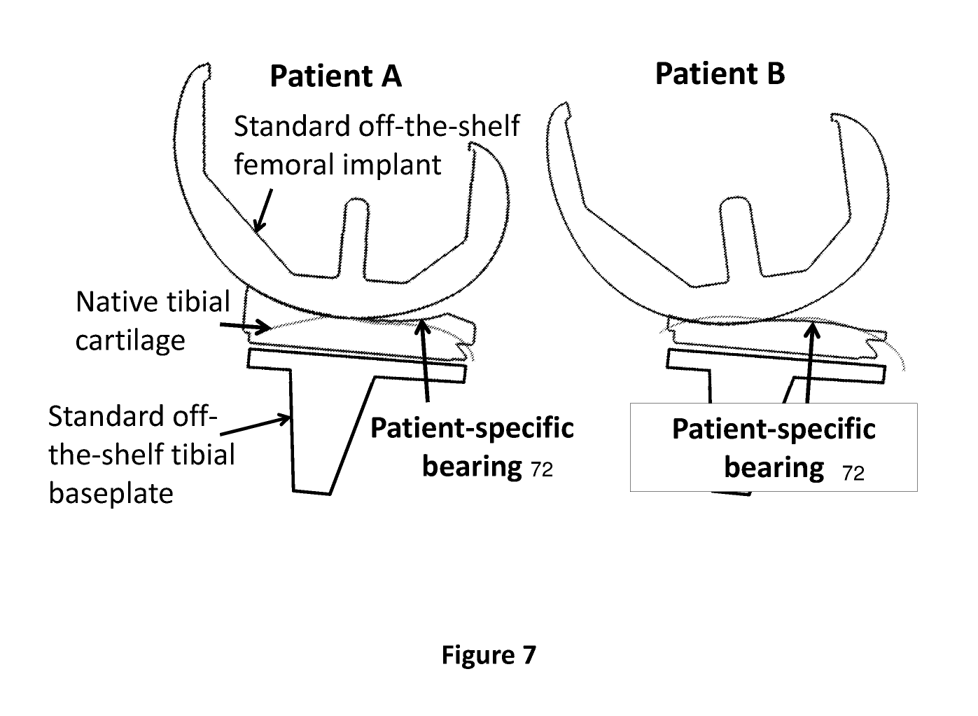

In TKR surgery two common types of implants are used; one that retains the posterior cruciate ligament (PCL), called cruciate retaining (CR) implant, and one that substitutes for the PCL through interaction between a femoral cam and a ligament replacement post on the tibial bearing, called posterior stabilized (PS) implant. In FIG. 8, the kinematics of a knee with two different CR bearing designs (patient-specific 82 and off-the-shelf/non-patient specific 84), articulating with the same off-the-shelf CR femoral and tibial baseplate are shown. In a normal knee we expect to see an overall medial pivot kinematics, with the lateral femoral condyle showing greater posterior motion than the medial condyle (7). This can be seen for the knee with the custom CR bearing. However, with the off-the-shelf bearing the knee shows kinematic abnormalities typical of off-the-shelf implants, including abnormal anterior sliding till 60° flexion (˜2 mm), minimal axial rotation (2° for off-the-shelf vs. 11.5° for patient-specific bearing), and reduced lateral rollback (˜7 mm less for off-the-shelf design relative to patient-specific design). This illustrates the potential benefit of utilizing the hybrid customization approach proposed here, involving combination of custom/patient-specific tibial bearings and off-the-shelf metal femoral components and tibial baseplate.

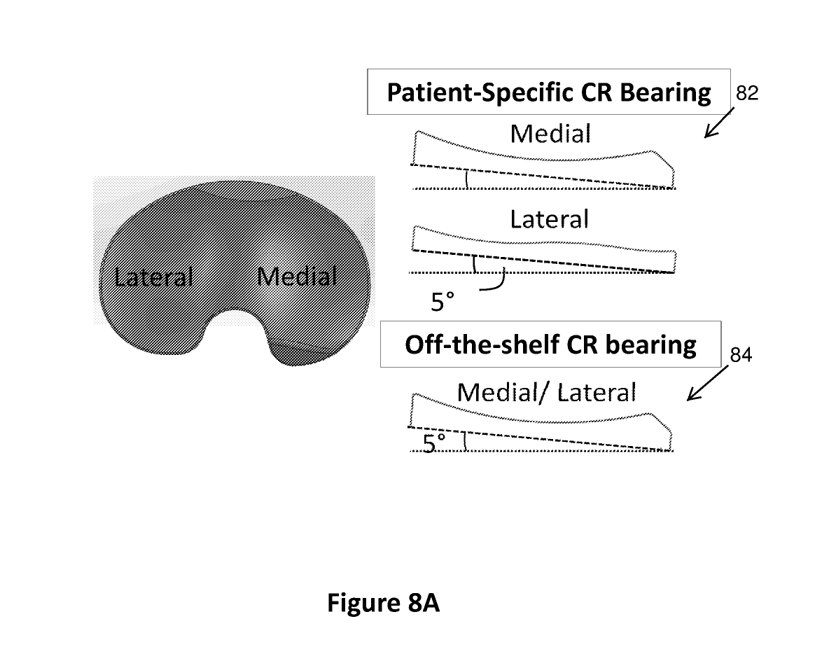

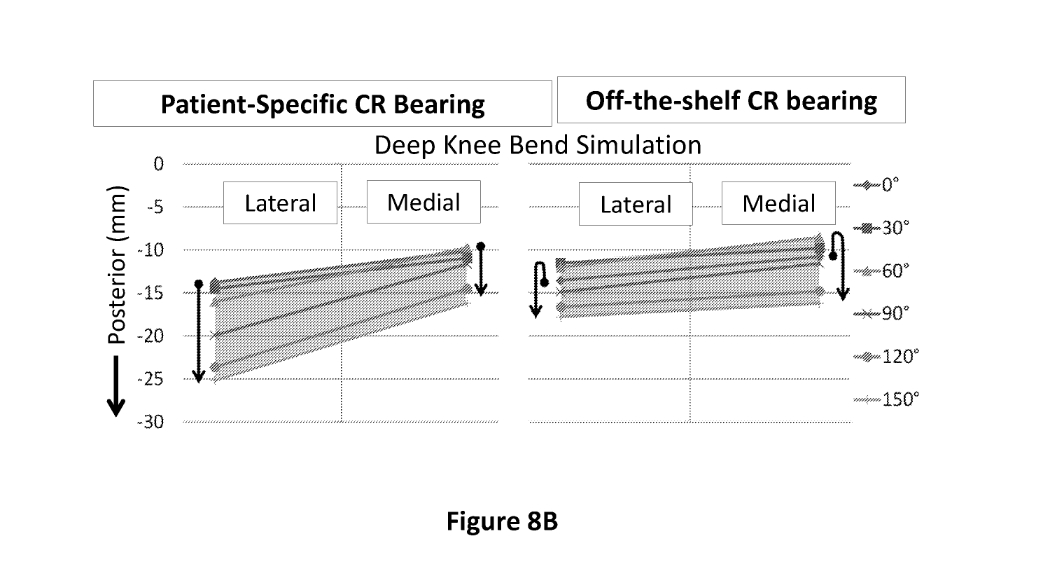

For a PS implant, not only can the articular surface of the tibial bearing be designed using the process shown in FIG. 4, but also the geometry and location of the tibial post can optimized for each patient. FIG. 9 shows how the process described in FIG. 4 can be used to create patient-specific PS tibial bearings compatible with off-the-shelf PS femoral components. In FIG. 10, the kinematics of a knee with two different PS tibial bearing designs (patient-specific 102 and off-the-shelf 104), articulating with the same off-the-shelf PS femoral are shown. With the off-the-shelf PS bearing, the knee shows several kinematic abnormalities, including excess posterior femoral location at 0° flexion, abnormal anterior sliding till 90° flexion (˜7 mm), and absence of medial rotation. In contrast, with the patient-specific PS bearing the knee shows more normal kinematics with anterior location of femoral condyles in extension (˜9 mm more anterior than off-the-shelf bearing), minimal anterior femoral sliding, and overall medial rotation characterized by greater posterior rollback of the lateral femoral condyle than the medial condyle (6). This further illustrates the potential benefit of utilizing the approach proposed here, involving combination of patient-specific tibial bearings and off-the-shelf femoral component and tibial baseplate.

In some embodiments, a method of manufacturing at least one component of a joint prosthesis is provided. The joint prosthesis can be a total knee replacement. The articulating bone can be a femur. The generic prosthetic component can be a generic femoral component. The articulating surface of the articulating bone can be a distal portion of a femur including a medial condyle and a lateral condyle. The kinematic data of the subject can include range of motion data between maximum flexion of the joint and maximum extension of the joint. The at least one component of the joint prosthesis can be a tibial bearing.

In some embodiments, the movement of the prosthetic articulating surface through the kinematic data of the subject through a bearing template can carve out the bearing surface of the kinematically appropriate bearing surface from the bearing template through a series of Boolean subtraction operations. An outer profile of the at least one component of the joint prosthesis can be trimmed according to the shape of a baseplate, and locking features are added on the component to mate with the baseplate.

In some embodiments, the method of manufacturing at least one component of a joint prosthesis can further comprise creating a fourth database representing stability data of a subject. The fourth database can be merged with the first database, the second database, and the third database representing kinematic data of the subject's joint. The prosthetic articulating surface can be stabilized corresponding to the stability data of the subject thereby modifying the bearing template to create a kinematically appropriate and stable bearing surface for the prosthetic articulating surface.

In some embodiments, the at least one component of the joint prosthesis can be machined to have a geometry corresponding to the modified bearing template having a kinematically appropriate bearing surface. The bearing surface can comprise at least one material selected from the group consisting of: polyaryletherketone (PEEK), polyolefins, polyethylene, ultra-high molecular weight polyethylene, medium-density polyethylene, high-density polyethylene, medium-density polyethylene, and highly cross-linked ultra-high molecular weight polyethylene (UHMWPE), or blends thereof.

### B: Geometric Approach to Designing Custom-Bearing Knee Implants

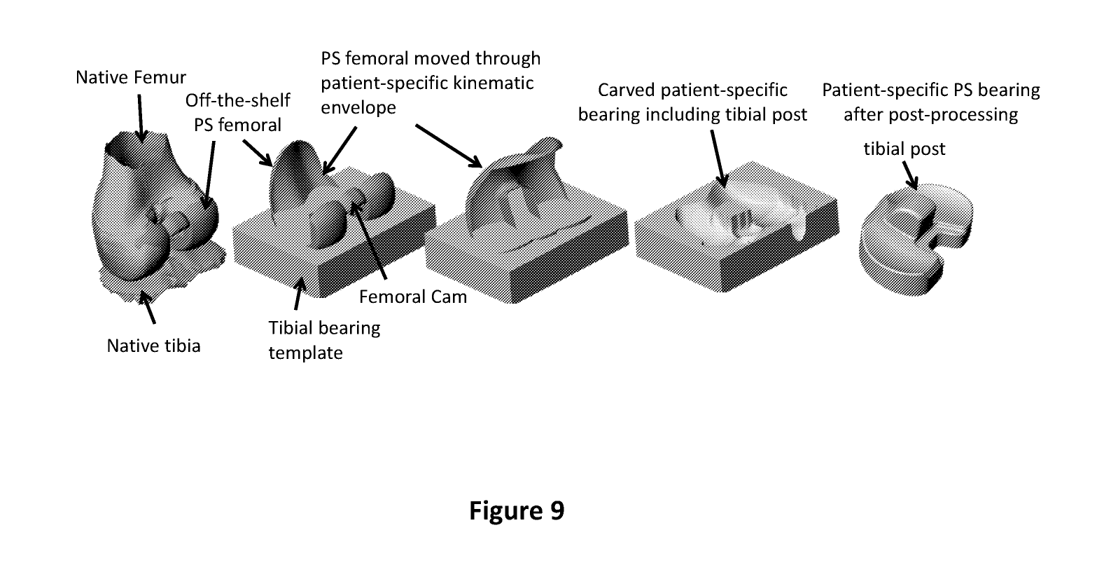

In another embodiment of the invention a geometric approach is taken for designing patient specific bearings. A 3D model of the patient's knee is created pre-operatively using CT, MRI, or other imaging modalities (FIG. 11A). Appropriately sized off-the-shelf femoral and tibial baseplate components are then mounted on the 3D model in a virtual environment (such as in a computer) in consultation with the surgeon. Alternatively, appropriately sized femoral and tibial baseplate components can be oriented in 3D space based on single-plane or multi-planar 2D images (such as radiographic images) of the patient's knee (Ref. 5, 6) (FIG. 11B). Following that, the relative orientation/position of the off-the-shelf femoral component relative to tibial baseplate is determined. Additionally, the differences between the articular geometry of femoral component (surface opposing to the bone facing side), and the patient's native femur are determined. These geometric differences and component position information are then used to design a custom-tibial bearing for the patient.

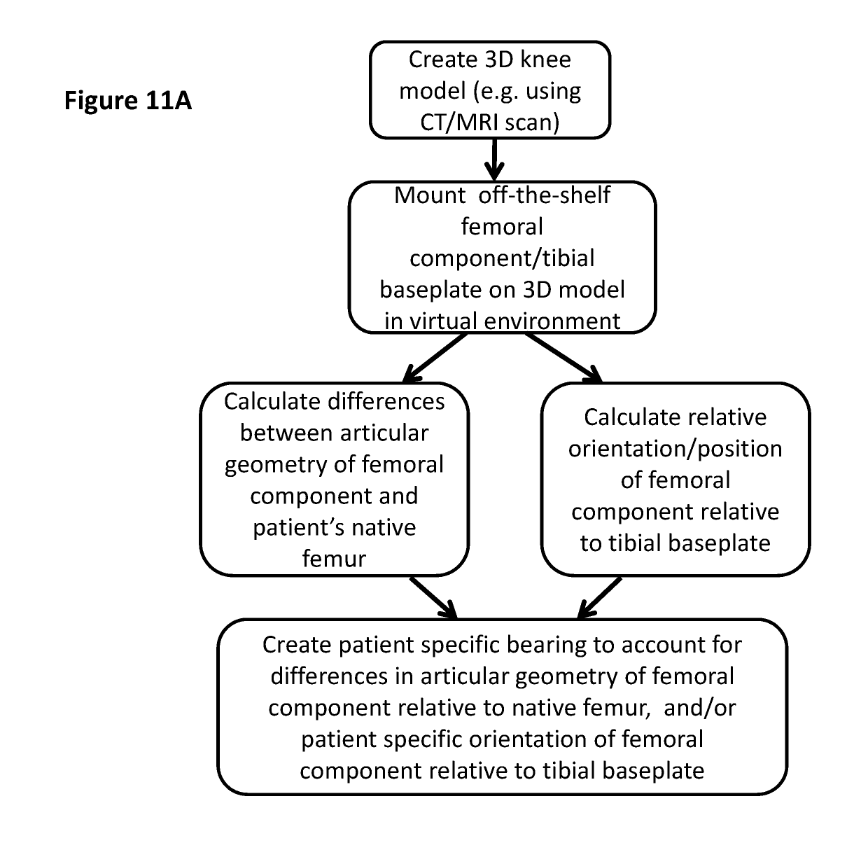

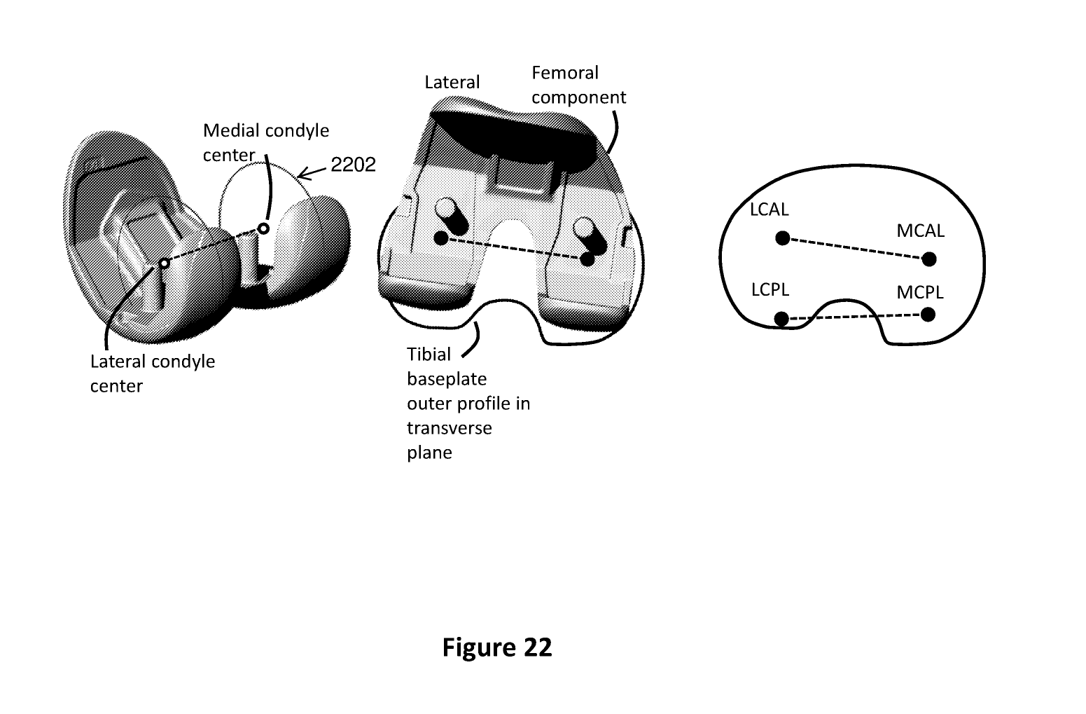

For example in FIG. 12, appropriate sized off-the-shelf femoral 122 and tibial baseplate 124 components are shown mounted on 3D knee models of 2 patients. The relative orientations of the femoral component and tibial baseplate, differ significantly between the two patients. The femoral component is externally rotated about 7.4° relative the tibial baseplate in patient A, vs. 15.2° in patient B. Therefore, in one embodiment of the invention, the low-point pathway of the tibial bearing (as seen in a transverse plane) is rotated 7.4° relative to an anteroposterior axis of the tibia in the patient-specific tibial bearing 126 for patient A, vs. 15.2° in the patient-specific tibial bearing 128 for patient B. Herein, low-point pathway refers to a line/curve joining the lowest points on the articular surface across a series of coronal cross-sections (refer to FIG. 25). Similarly, in patient A, the femoral component 132 is medially shifted by ˜4.8 mm relative to the tibial baseplate 134 vs. 7 mm in patient B (FIG. 13). Therefore, in one embodiment of the invention the low-point pathway seen in a transverse plane is shifted medial by ˜4.8 mm relative to a mid-plane of the tibia in the patient-specific tibial bearing for patient A vs. an ˜7 mm in the patient-specific tibial bearing for patient B.

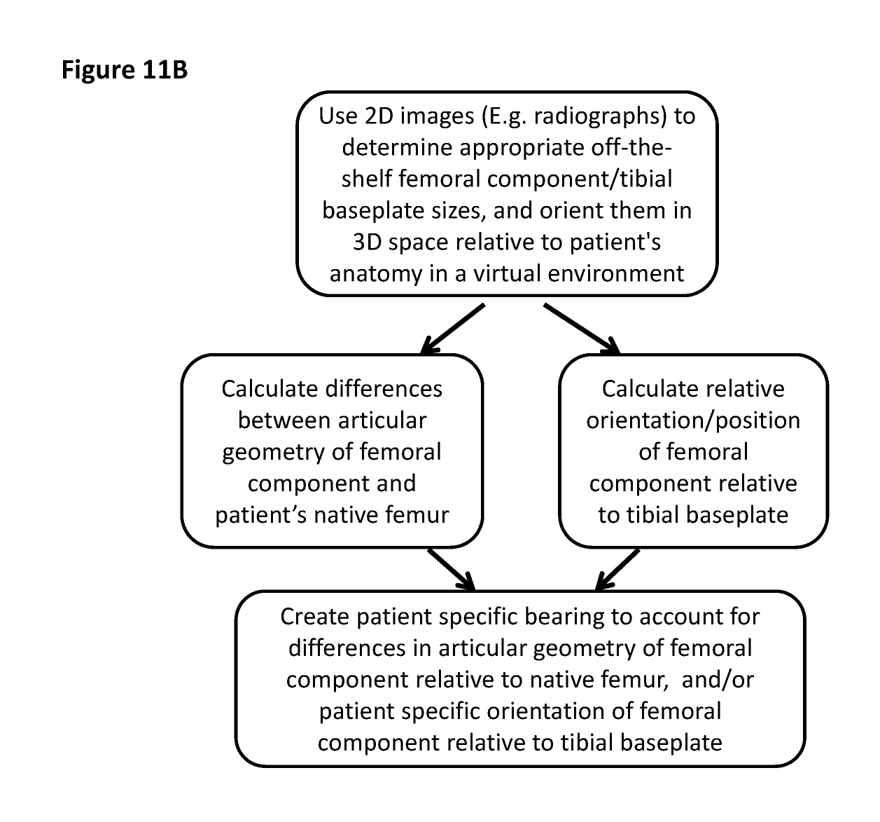

In FIG. 14A, sagittal plane profile of an off-the-shelf femoral component 1402 mounted on the native femur 1404 of a patient is shown. As evident from the figure, the articular surface of the femoral component closely matches the articular surface of the native femur (bone/cartilage) in regions 2, 3, and 4. However, the femoral component's articular surface is shifted inward (i.e. towards the interior of the femur) relative to that of the native femur in regions 1 and 5. To compensate for this difference, the custom tibial bearing 1410, 1412 for this patient is modified in regions 1 and 5 to be more proximal relative to off-the-shelf tibial insert designs 1414, 1416 (FIGS. 14B, C).

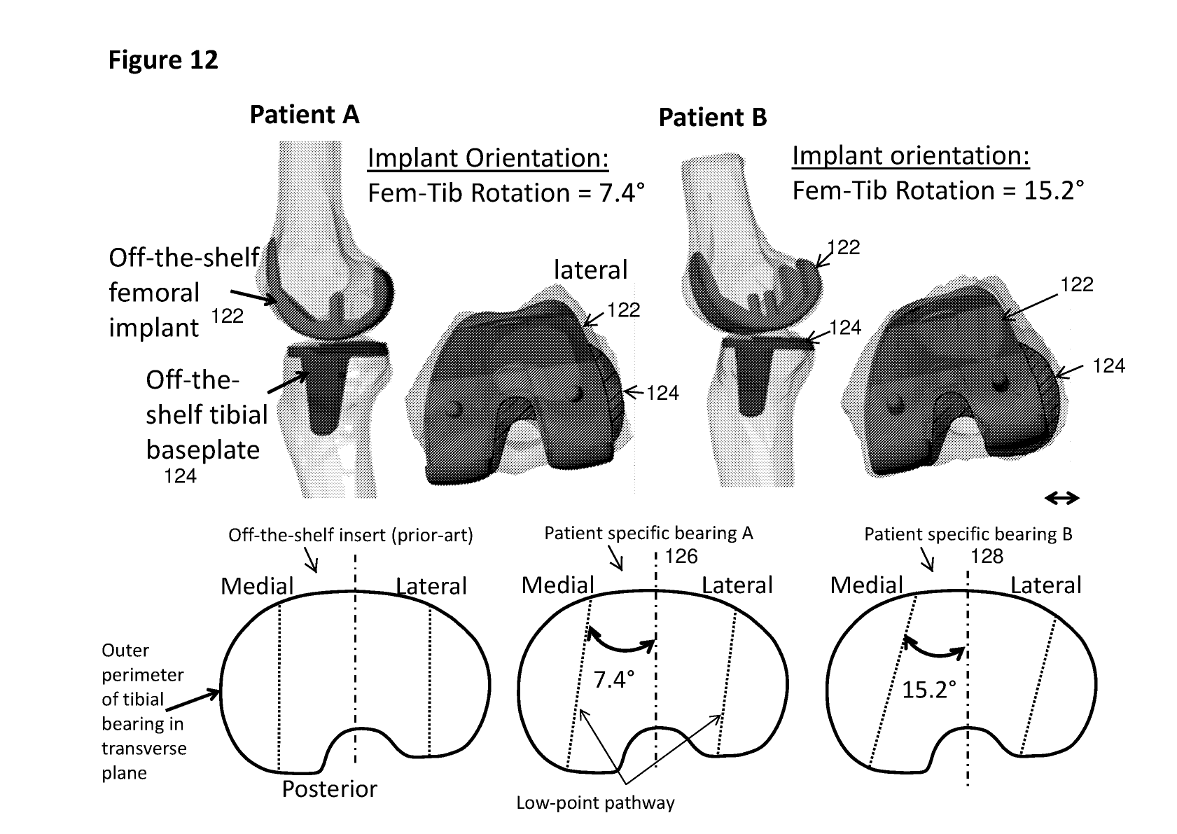

In FIG. 15A, sagittal plane profile of an off-the-shelf femoral component 1502 mounted on the native femur 1504 of another patient is shown. As seen from the figure, the articular surface of the femoral component closely matches the articular surface of the native femur in regions 2, 3, and 4. However, the femoral component's articular surface is shifted inward (i.e. towards the interior of the femur) relative to that of the native femur in region 1, and is shifted outward relative to native femur in region 5. To compensate for this difference, the custom tibial bearing 1510, 1512 for this patient is modified to be more proximal relative to off-the-shelf tibial insert designs in region 1, and to be more distal relative to off-the-shelf tibial insert designs 1514, 1516 in region 5 (FIGS. 15B, C).

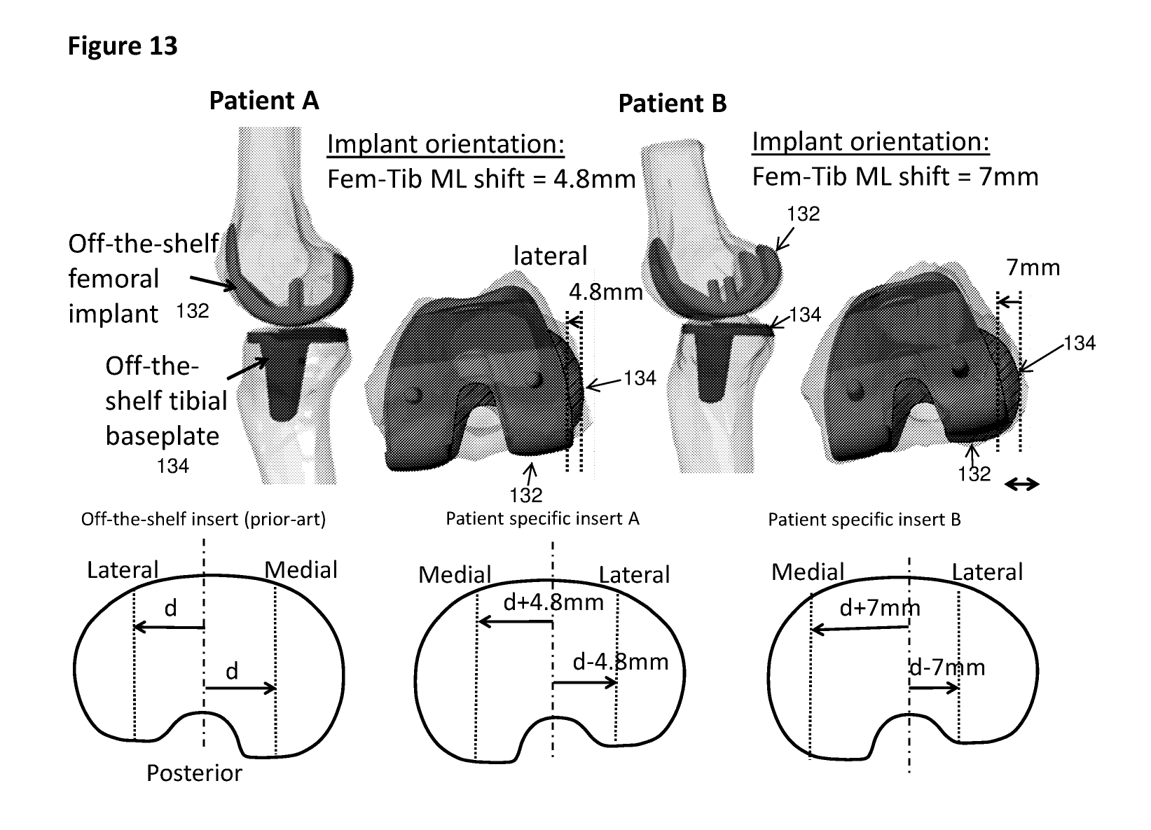

In FIG. 16A, sagittal plane profile of an off-the-shelf femoral component 1602 mounted on the native femur 1604 of another patient is shown. As seen from the figure, the articular surface of the femoral component closely matches the articular surface of the native femur in regions 2, and 4. However, the femoral component's articular surface is shifted inward (i.e. towards the interior of the femur) relative to that of the native femur in region 1, 3 and 5. To compensate for this difference, the custom tibial bearing 1610, 1612 for this patient is modified to be more proximal relative to off-the-shelf tibial insert designs 1614, 1616 in region 1, 3 and 5 (FIGS. 16B, C).

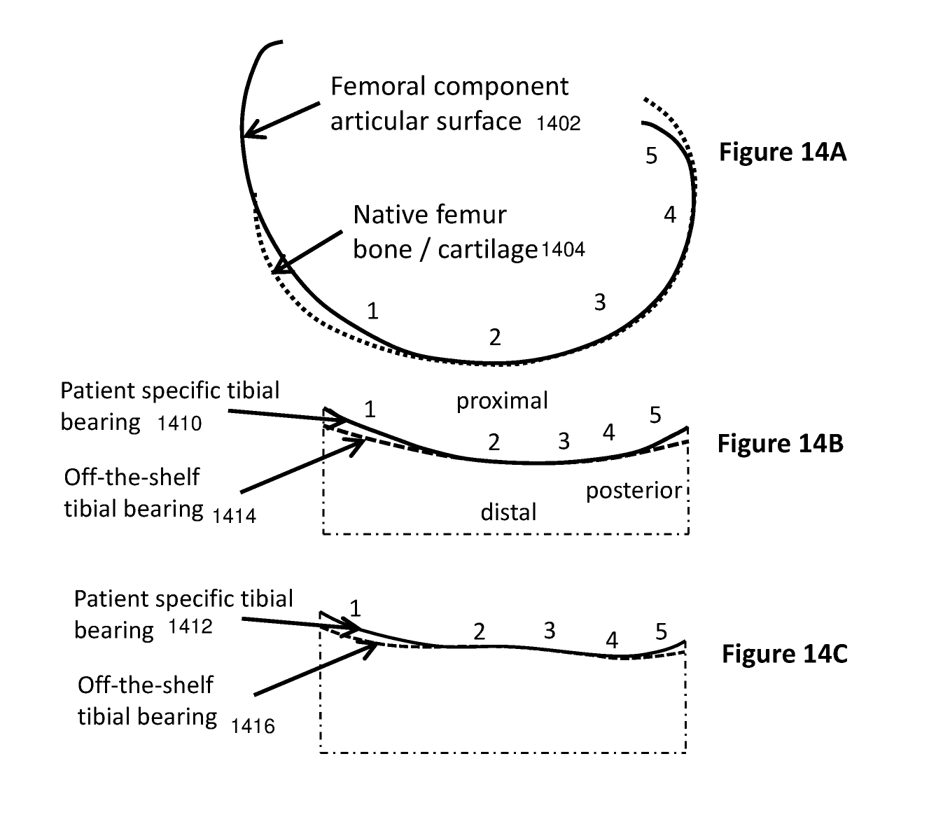

In FIG. 17A, sagittal plane profile of an off-the-shelf femoral component 1702 mounted on the native femur 1704 of another patient is shown. As seen from the figure, the articular surface of the femoral component closely matches the articular surface of the native femur in regions 2 and 4. However, the femoral component's articular surface is shifted inward (i.e. towards the interior of the femur) relative to that of the native femur in region 1 and 3, and shifted outwards relative to native femur in region 5. To compensate for this difference, the custom tibial bearing 1710, 1712 for this patient is modified to be more pronounced relative to off-the-shelf tibial insert designs 1714, 1716 in regions 1 and 3, and to be more distal relative to off-the-shelf tibial insert designs 1714, 1716 in region 5 (FIGS. 17B, C).

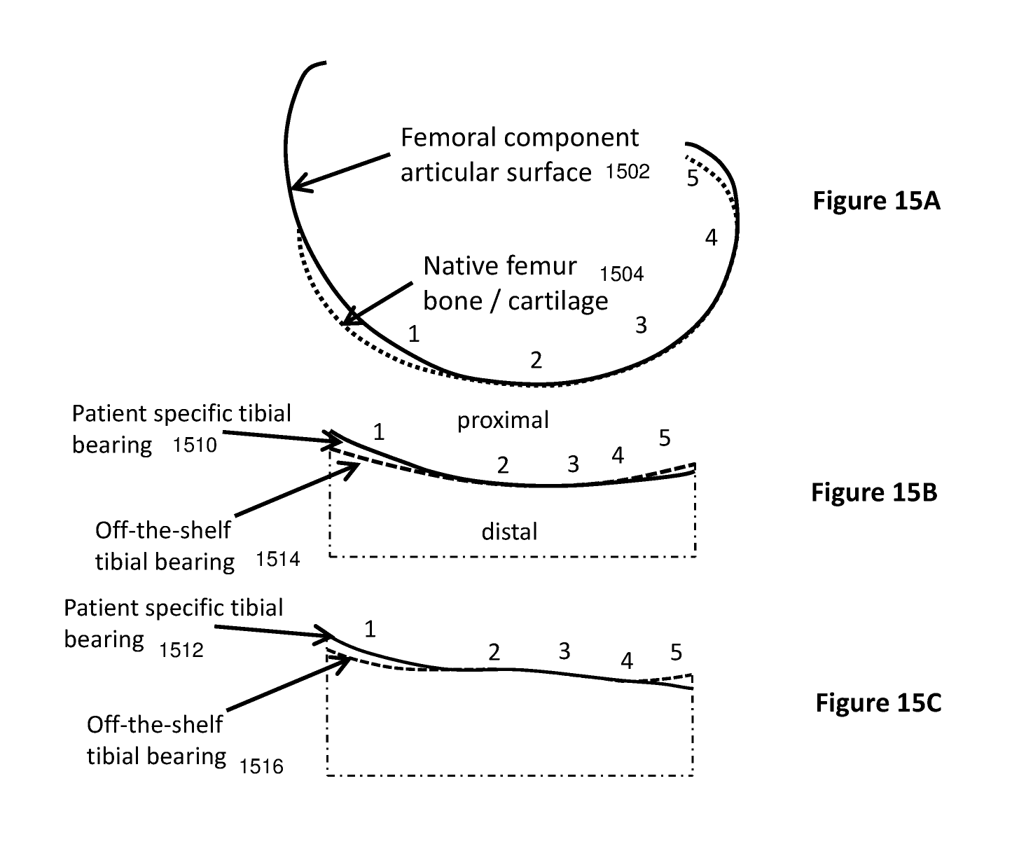

In FIG. 18A, sagittal plane profile of an off-the-shelf femoral component 1802 mounted on the native femur 1804 of another patient is shown. As seen from the figure, the articular surface of the femoral component closely matches the articular surface of the native femur in regions 2, 4, and 5. However, the femoral component's articular surface is shifted inward relative to that of the native femur in region 1 and 3. To compensate for this difference, the custom tibial bearing 1710, 1712 for this patient is modified to be more proximal relative to off-the-shelf tibial insert designs 1714, 1716 in regions 1 and 3 (FIGS. 18B, C).

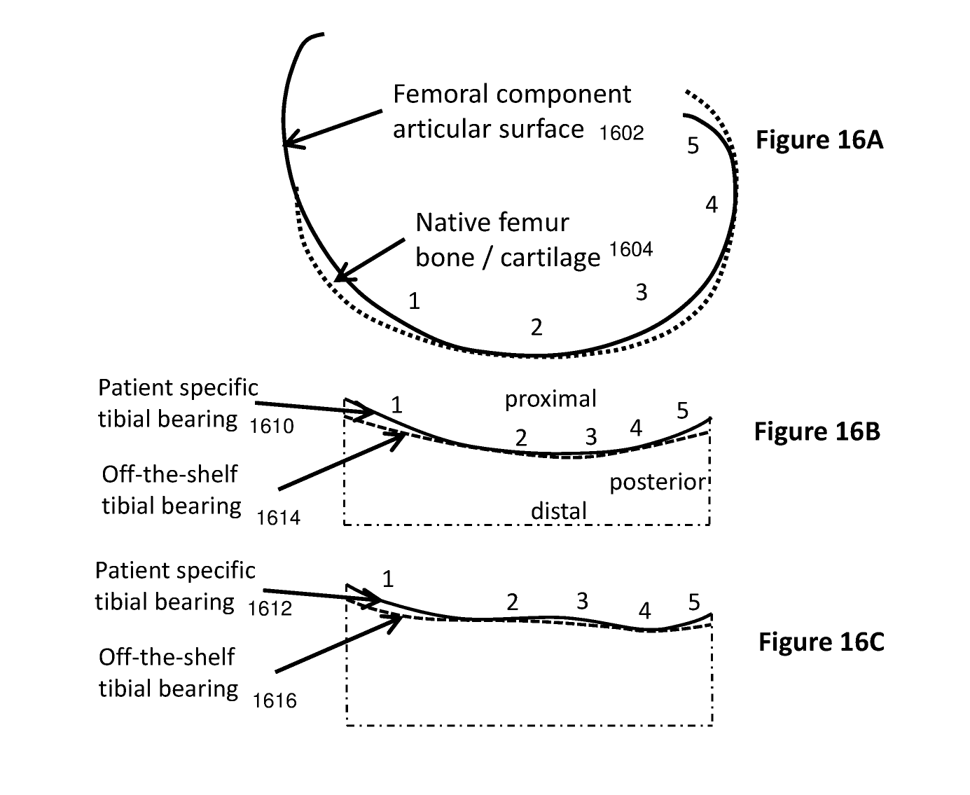

In FIG. 19A, sagittal plane profile of an off-the-shelf femoral component 1902 mounted on the native femur 1904 of another patient is shown. As seen from the figure, the articular surface of the femoral component closely matches the articular surface of the native femur in regions 2, 4, and 5. However, the femoral component's articular surface is shifted inwards relative to that of the native femur in region 1 and is shifted outwards relative to that of the native femur in region 3. To compensate for this difference, the custom tibial bearing 1910, 1912 for this patient is modified to be more proximal relative to off-the-shelf tibial insert designs 1914, 1916 in region 1 and more distal relative to off-the-shelf tibial insert designs 1914, 1916 in region 3 (FIGS. 19B, C).

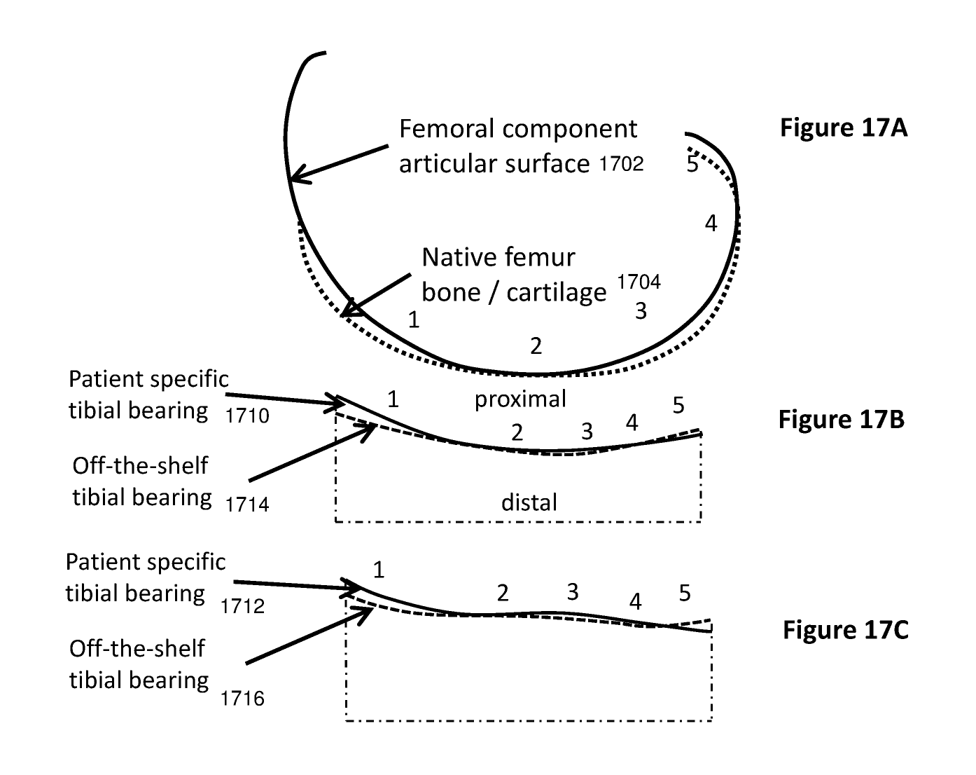

In FIG. 20A, sagittal plane profile of an off-the-shelf femoral component 2002 mounted on the native femur 2004 of another patient is shown. As seen from the figure, the articular surface of the femoral component closely matches the articular surface of the native femur in regions 2 and 4. However, the femoral component's articular surface is shifted outwards relative to that of the native femur in region 1, and shifted inwards relative to native femur in regions 3 and 5. To compensate for this difference, the custom tibial bearing 2010, 2012 for this patient is modified to be more distal relative to off-the-shelf tibial insert designs 2014, 2016 in region 1, and to be more proximal relative to off-the-shelf tibial insert designs 2014, 2016 in regions 3 and 5 (FIGS. 20B, C).

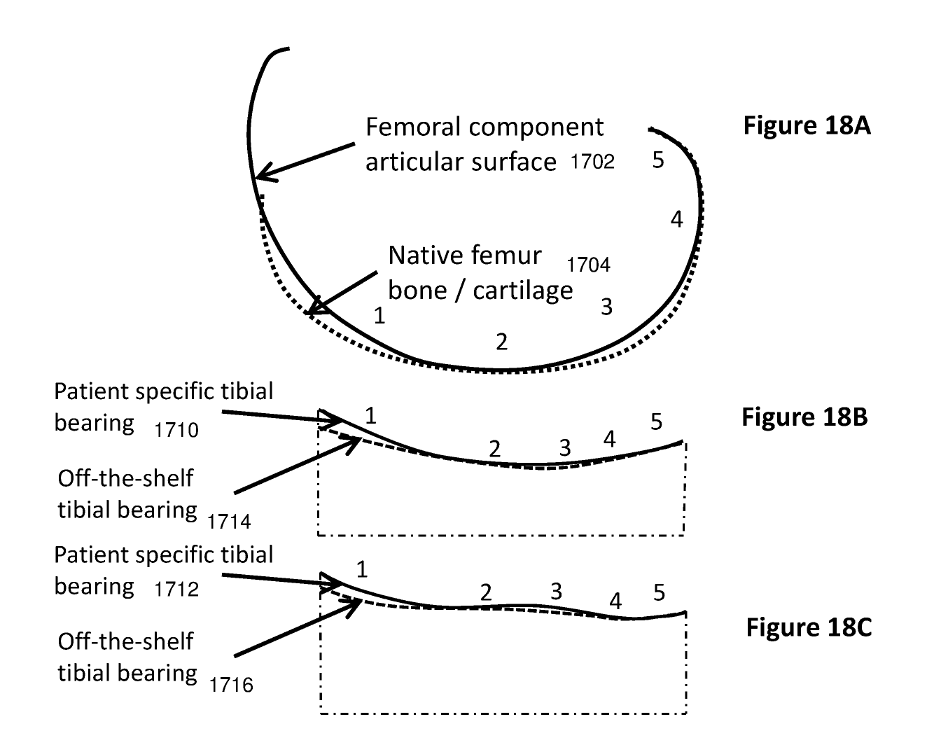

In FIG. 21A, sagittal plane profile of an off-the-shelf femoral component 2102 mounted on the native femur 2104 of another patient is shown. As seen from the figure, the articular surface of the femoral component closely matches the articular surface of the native femur in regions 2 and 4. However, the femoral component's articular surface is shifted outwards relative to that of the native femur in regions 1 and 5, and shifted inwards relative to native femur in region 3. To address this difference, the custom tibial bearing 2110, 2112 for this patient is modified to be more distal relative to off-the-shelf tibial insert designs 2114, 2116 in regions 1 and 5, and to be more proximal relative to off-the-shelf tibial insert designs 2114, 2116 in region 3 (FIGS. 21B, C).

In some embodiments, the method of manufacturing at least one component of a joint prosthesis is provided. The joint prosthesis can be a total knee replacement. The first articulating bone can be a femur. The first generic prosthetic component can be a generic femoral component. The first articulating bone can have an articulating surface that is a distal portion of a femur including a medial condyle and a lateral condyle. The custom prosthetic component can be a tibial bearing. The custom prosthetic component can comprise at least one material selected from the group consisting of polyaryletherketone (PEEK), polyolefins, polyethylene, ultra-high molecular weight polyethylene, medium-density polyethylene, high-density polyethylene, medium-density polyethylene, highly cross-linked ultra-high molecular weight polyethylene (UHMWPE), and blends thereof. The surface contour deviation can represent one or more of an inward shift and an outward shift of the first prosthetic articulating surface from the outer articulating surface of the articulating bone.

### Selection of Anatomically and Kinematically Appropriate Tibial Bearing Surface

Patient-specific bearings designed for different patients may differ in one or more ways including geometry of medial/lateral articular surface, proximal-distal thickness of medial/lateral compartment, geometry/location of tibial post, posterior slopes of the medial/lateral compartment, coronal plane slope of the medial/lateral compartment. Such bearings may be designed for either total and partial knee joint replacement. As an alternative to machining bearings for each individual patient, the most appropriate bearing geometry for a given patient can also be selected from a library of bearings. Such library of bearings may be designed to accommodate or provide different knee kinematic pattern/s. Irrespective of whether the bearing is machined specifically for a given patient, or selected from a library of bearings, the geometry of bearings can take the form of one or more embodiments described below, or a combination thereof.

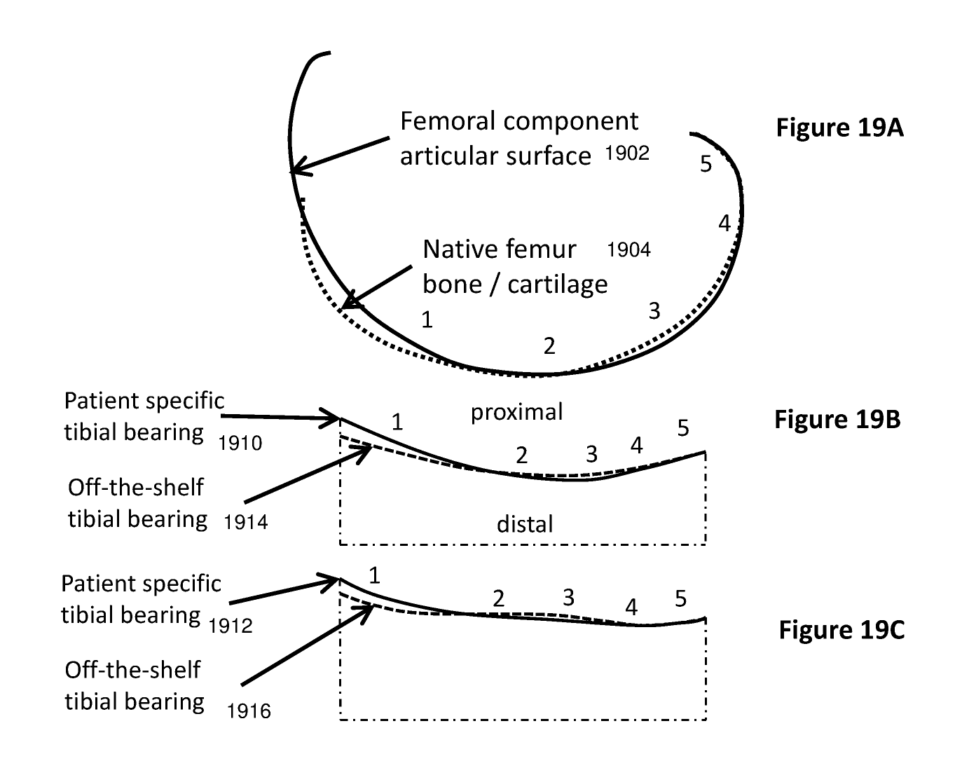

The tibiofemoral kinematics of a knee can be defined in a variety of different ways, such as motion of femur relative to tibia or tibia relative to femur. One way of describing tibiofemoral kinematics is to describe motion of the medial and lateral femoral condyle centers in three-dimensional space relative to a tibial coordinate system 2202 (FIG. 22). Focusing on motion in a transverse plane, the location of femur relative to tibia at a given knee flexion angle, can be fully characterized by 3 parameters (AP location, ML location, and internal-external rotation). Ignoring the mediolateral motion for illustration purposes, the motion of the femur relative to tibia in a transverse plane between two knee flexion angles, can be defined by 4 parameters, with 2 parameters describing femur location at the first flexion angle and 2 parameters describing femur location at the second flexion angle. For example, the anteroposterior location and internal-external femoral rotation at full extension and maximum flexion can be fully described with 4 parameters—medical condyle anterior location at full knee extension (MCAL), medial condyle posterior location at maximum knee flexion (MCPL), lateral condyle anterior location at full extension (LCAL), and lateral condyle posterior location at maximum flexion (LCPL) (FIG. 22). The same motion can be also be described using different set of 4 independent parameters, for e.g. anterior location of femur center (point mid-way between line joining medial/lateral condyle) at full extension, posterior location of femur center at maximum flexion, femoral IE rotation at full extension, and femoral IE rotation at maximum knee flexion.

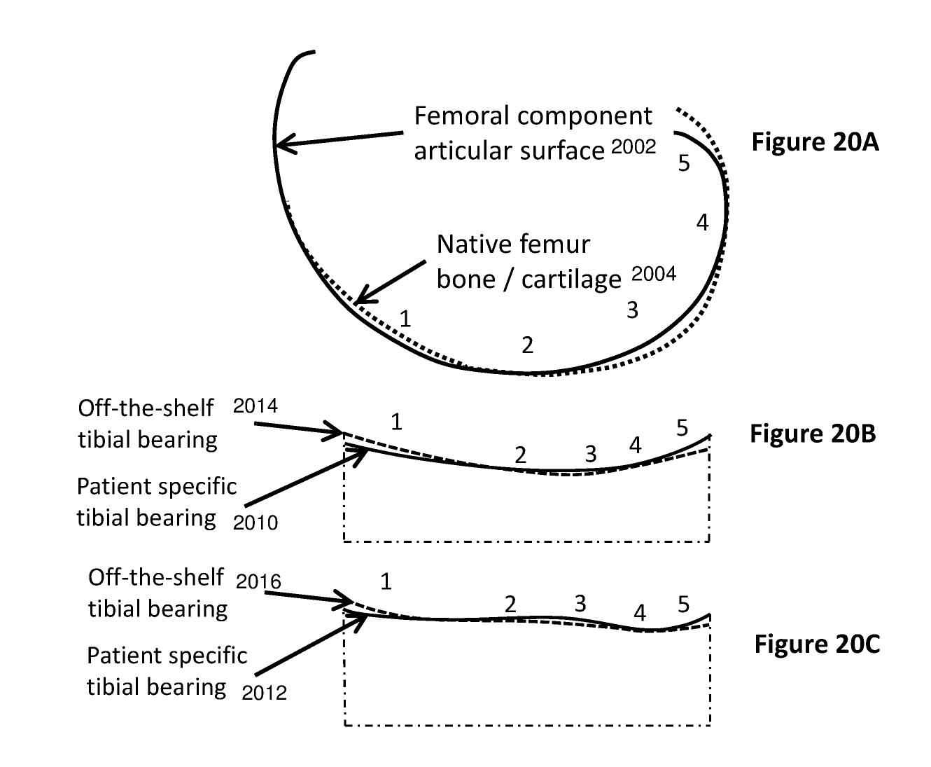

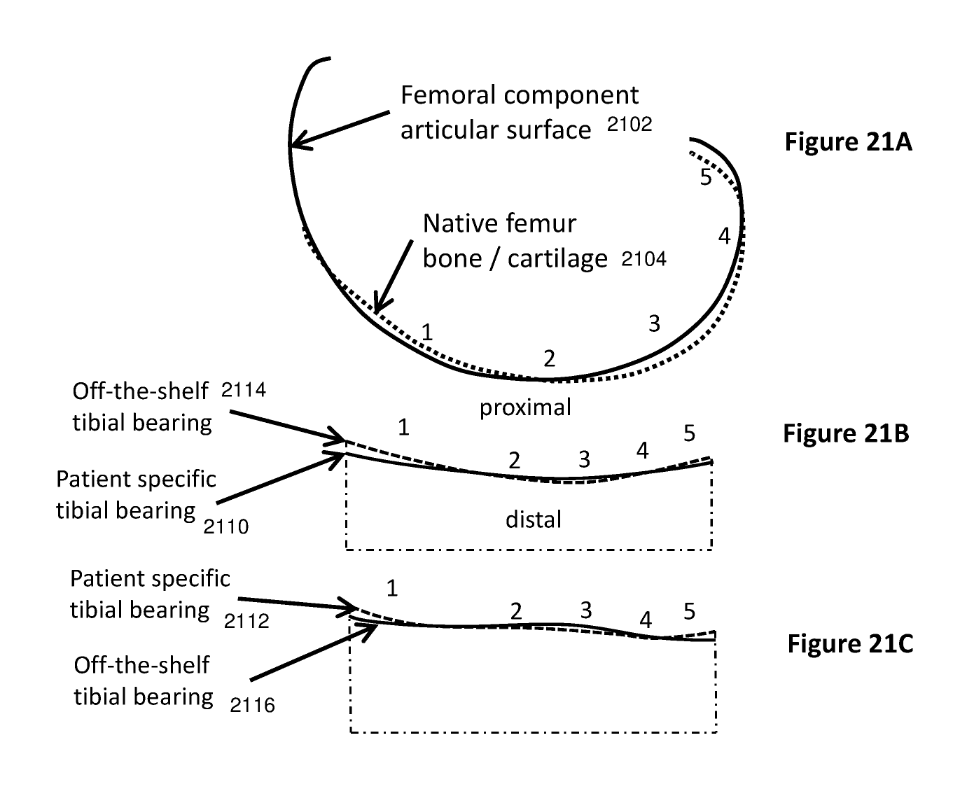

In one embodiment of the invention, for a given bearing size (AP size, ML size) and PD thickness, a library of tibial bearings with different articular geometries is provided. This library of bearings is designed to provide different tibiofemoral kinematic patterns, from which the most appropriate bearing can be selected for each patient. For example, a default tibial bearing “0000” may be designed to provide a default kinematic pattern as shown in FIG. 23 (Bearing A-“0000”). Additional bearings in the library could be designed to provide more anterior medial/lateral condyle location (denoted by “+” symbol), more posterior medial/lateral condyle location (denoted by “+” symbol), less anterior medial/lateral condyle location (denoted by corresponding “−” symbol), less posterior medial/lateral condyle location (denoted by corresponding “−” symbol), relative to the default bearing (FIGS. 23, 24). In one embodiment of the invention, 81 bearings provide 81 different kinematic patterns, (4 kinematic parameters—MCAL, LCAL, MCPL, LCPL; 3 levels of each parameter—“0”, “+”, “−”; =>3⁴). In other embodiments, a library of bearings could be designed based on n kinematic parameters (n=1, 2, 3 . . . etc.), with each parameter having k levels (k=1, 2, 3, 4, etc.). The number of total bearings in the library can be reduced by having separate medial and lateral bearing components. For example, 9 medial bearings designed to provide 3 different levels of MCAL and MCPL (i.e. medial bearings “00”, “0+” etc.), can be combined with 9 lateral bearings designed to provide 3 different levels of LCAL and LCPL (i.e. lateral bearings “00”, “0+” etc.), to obtain 81 unique combinations (e.g. bearing “++−−”=medial bearing “++” combined with lateral bearing “−−”).

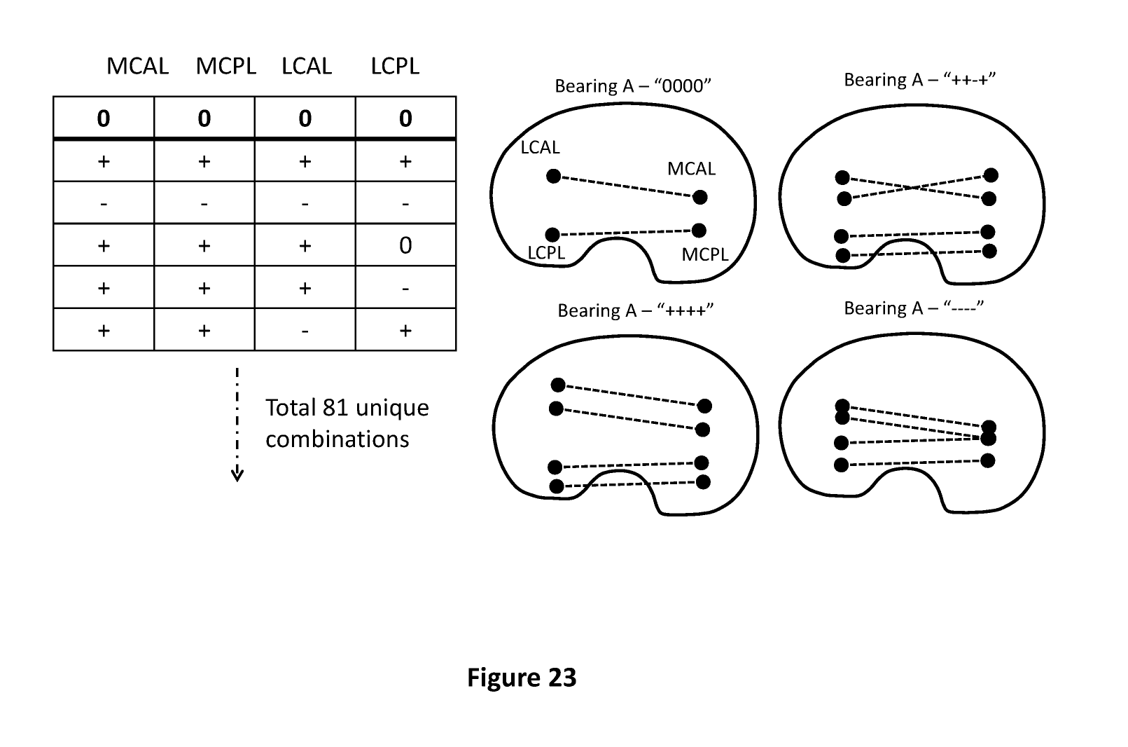

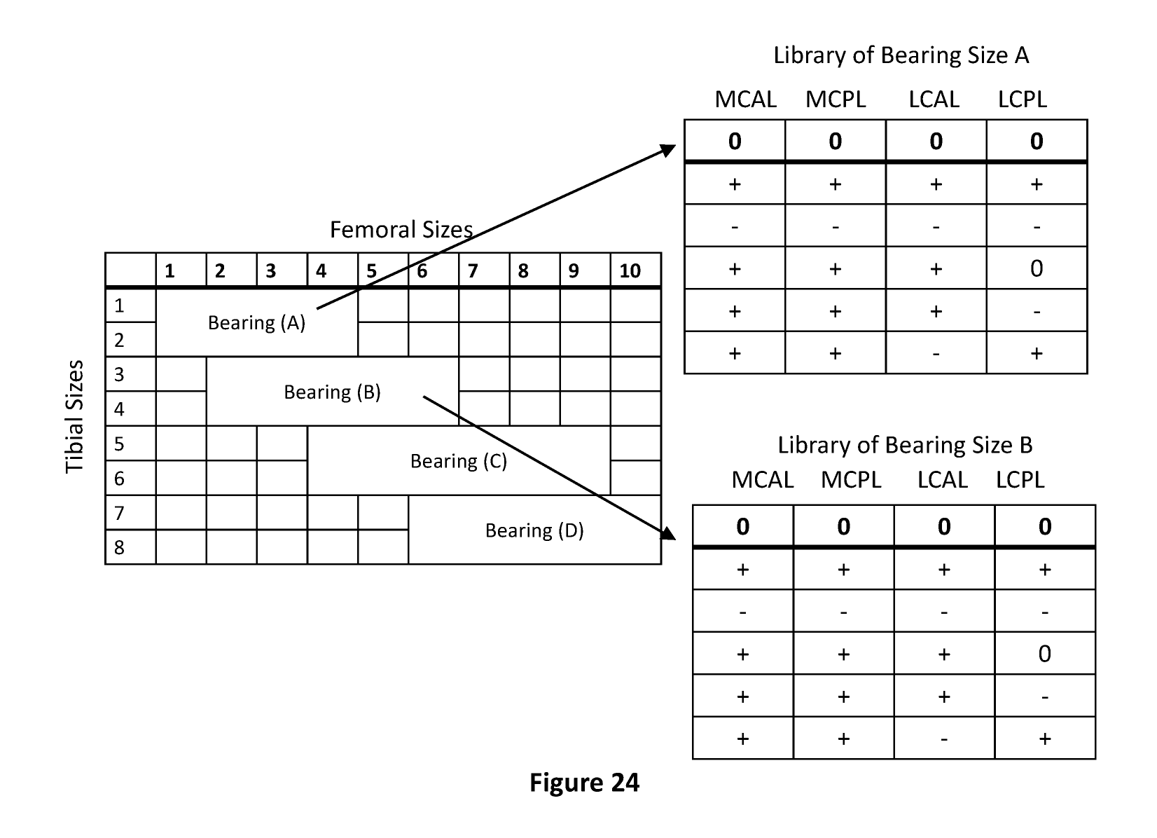

The tibial bearings in the library can be designed with different geometries in the transverse plane, sagittal plane and/or coronal plane to provide different tibiofemoral kinematics. In some embodiments of the invention the medial and lateral tibial low-point pathway/s (LP/s) can be varied to provide different kinematic patterns (FIG. 25). For example, in the embodiment of a tibial bearing 2620 having bearing surface 2622 as shown in FIG. 26B, the lateral LP is parallel to the AP axis in a transverse plane, while the medial LP is angled. In other embodiments, a portion of the medial or lateral LP can be angled relative to a bearing AP axis in a transverse plane, while other portions can lie along straight lines parallel to the said AP axis. For example in embodiment of a tibial bearing 2630 having bearing surface 2632 as shown in FIG. 26C, anterior portions of medial and lateral LPs are angled towards the medial side of the knee relative to a bearing AP axis, while the posterior portion of medial and lateral LPs are parallel to the said AP axis. In embodiment of a tibial bearing 2640 having bearing surface 2642 as shown in FIG. 26D, anterior portion of the lateral LP is angled towards the medial side of the knee relative to a bearing AP axis, while the posterior portion of medial and lateral LPs are parallel to the said AP axis. In other embodiments, some portions of the medial and/or lateral LPs may be curved in a transverse plane while other portions might be straight. For example, in embodiment of a tibial bearing 2650 having bearing surface 2652 as shown in FIG. 26E the anterior and posterior portions of medial and lateral LPs are curved while the central portions are straight and parallel to a bearing AP axis. In this embodiment, the anterior portions of medial and lateral LPs are curved by different amounts with the concave side of the curves facing medially. Further, in this embodiment, the posterior portions of medial and lateral LPs are curved by different amounts with the concave side of the curves facing laterally. In embodiment of a tibial bearing 2710 having bearing surface 2712 as shown in FIG. 27A, the anterior portions of medial and lateral LPs are angled laterally relative to a bearing AP axis while the posterior portions are parallel to the said bearing AP axis. In embodiment of a tibial bearing 2720 having bearing surface 2722 as shown in FIG. 27B, the anterior portions of medial and lateral LPs are curved in a transverse plane with the concave side of the curve facing laterally, and the posterior portions of medial and lateral LPs are curved with the concave side of the curve facing medially.

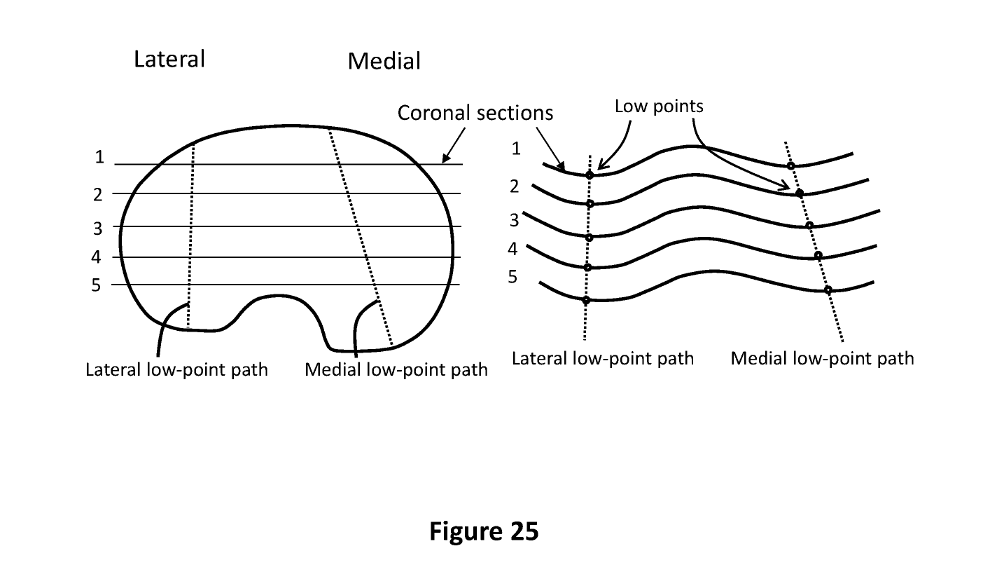

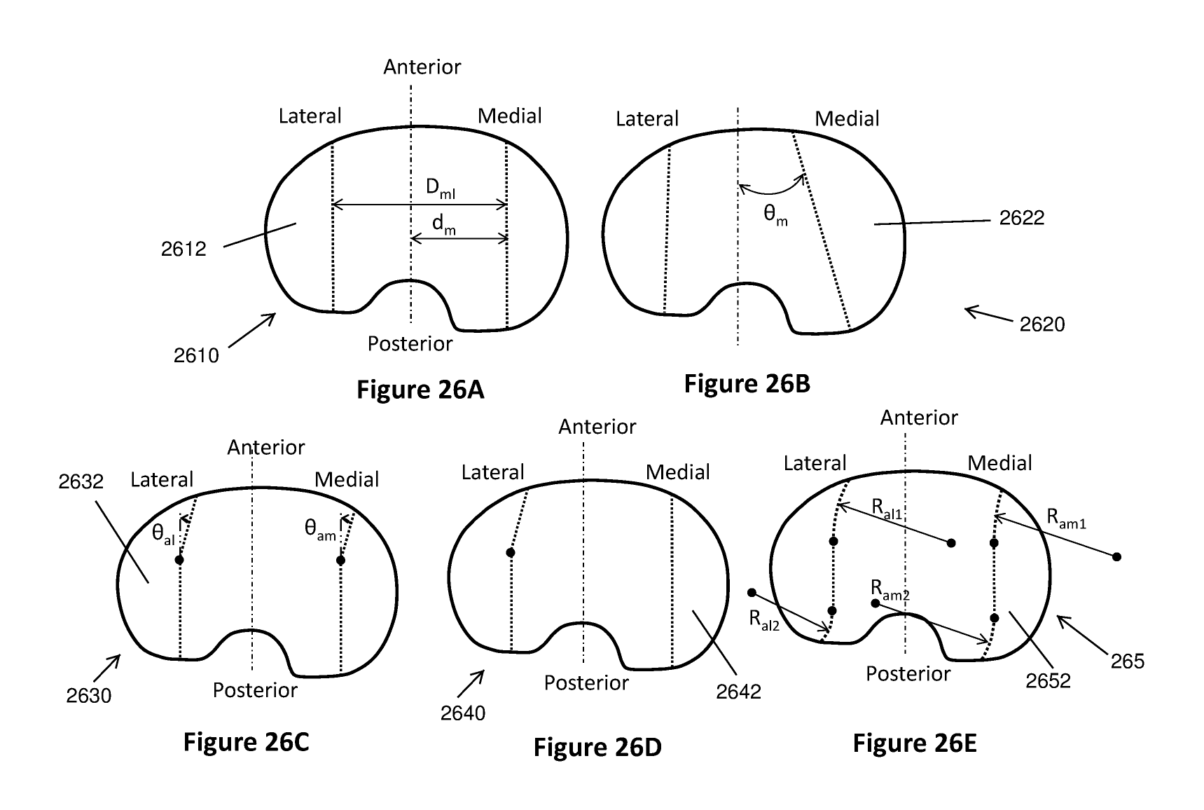

In other embodiments of the invention bearings may be provided with medial and/or lateral LPs curved in a transverse plane. The curvature or the location of the center of curvature of the medial/lateral LPs can be varied across the bearings in the library. For example, in embodiment of a tibial bearing 2810 having bearing surface 2812 as shown in FIG. 28A, both medial and lateral LPs are curved with the concave side of the curvature facing medially. Further in this embodiment the center of curvature is different for the medial LP and lateral LP, and located outside the tibial bearing. In embodiment of a tibial bearing 2820 having bearing surface 2822 as shown in FIG. 28B, both medial and lateral LPs are curved with the concave side of the curvature facing medially, and both having same center of curvature. In embodiment of a tibial bearing 2830 having bearing surface 2832 as shown in FIG. 28C, both medial and lateral LPs are curved with the concave side of the curvature facing medially, and both having different centers of curvature. In this embodiment the medial LP center of curvature is located outside the tibial bearing, while the lateral LP center of curvature is located within the tibial bearing. In other embodiments, both medial and lateral LPs can be curved with the concave side of the curvature facing laterally. In embodiment of a tibial bearing 2910 having bearing surface 2912 as shown in FIG. 29A, both medial and lateral LPs are curved with the concave side of the curvature facing laterally, and both having different centers of curvature located outside the tibial bearing. In embodiment of a tibial bearing 2920 having bearing surface 2922 as shown in FIG. 29B, both medial and lateral LPs are curved with the concave side of the curvature facing laterally, and both having the same center of curvature.

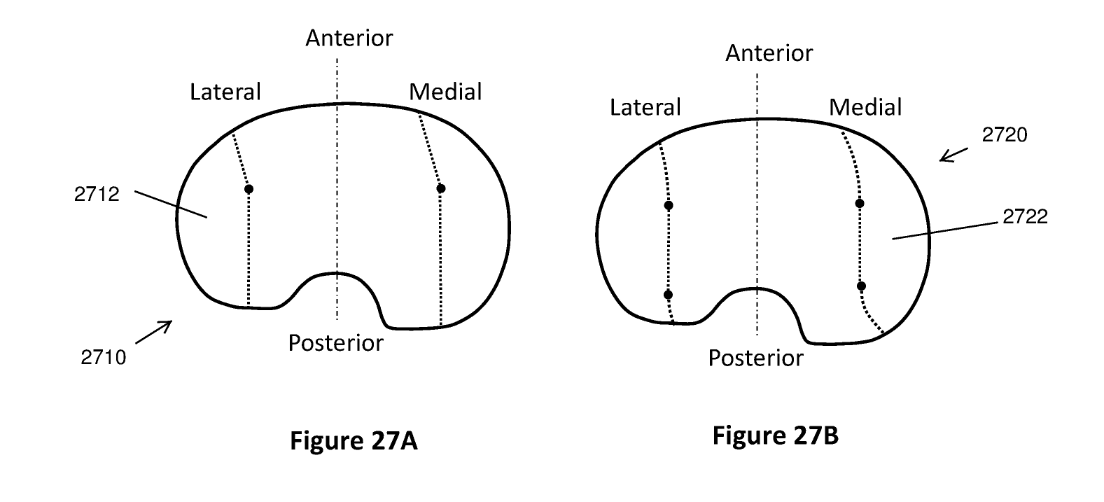

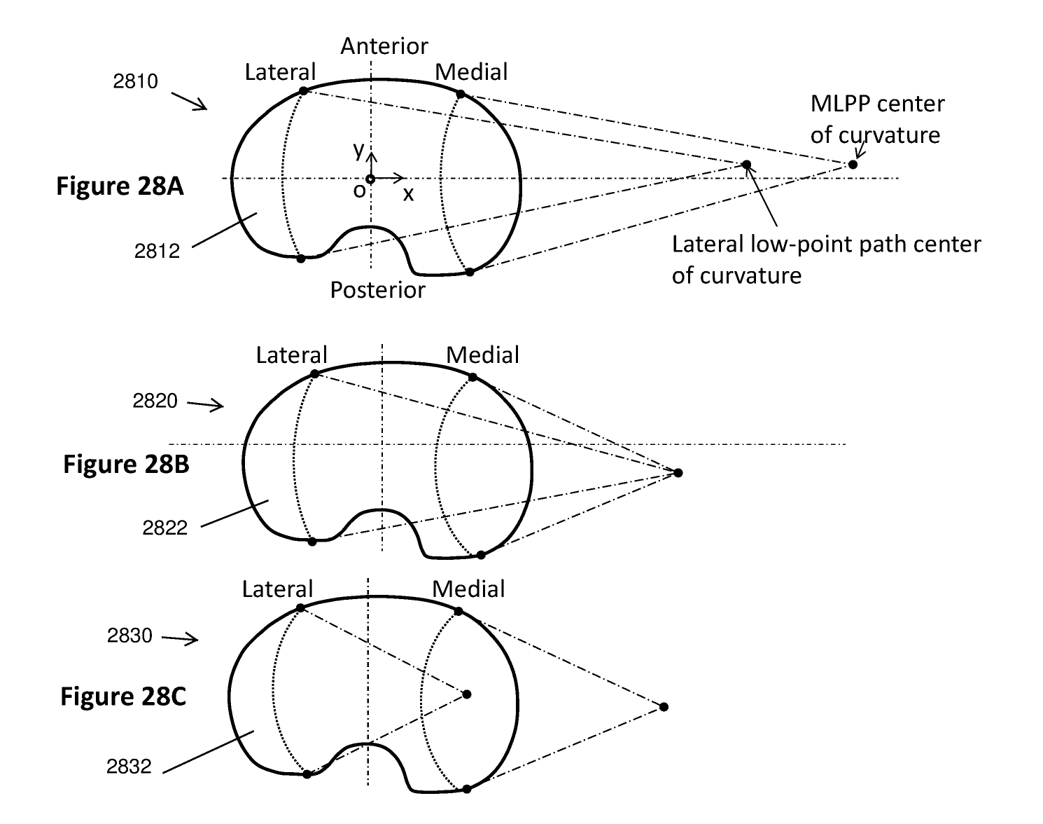

In other embodiments, the orientation of medial and lateral LPs may be varied across the tibial bearings in the library. For example, FIGS. 30A to 30E show different bearings (3010, 3020, 3030, 3040, 3050, 3060) of the same size from the library with bearing surfaces (3012, 3022, 3032, 3042, 3052, 3062) having medial and lateral LPs lying along straight lines in a transverse plane, but with the LPs angled by different amounts and/or in different directions amongst the different bearings relative to a bearing AP axis. In another embodiment the orientation of different portions of the medial and lateral LPs may be varied across the tibial bearings in the library. For example, FIGS. 31A to 31E shows different bearings of the same size from another embodiment of the library. In embodiment of a tibial bearing 3110 having bearing surface 3112 as shown in FIG. 31A the medial and lateral LPs lie along straight lines parallel to a bearing axis, in embodiment of a tibial bearing 3120 having bearing surface 3122 as shown in FIG. 31B the anterior medial and lateral LPs are angled relative to the bearing axis, in embodiment of a tibial bearing 3130 having bearing surface 3132 as shown in FIG. 31C the anterior medial and lateral LPs are angled by greater amount relative to the bearing axis than that in bearing of FIG. 31B, and in embodiment of a tibial bearing 3140 having bearing surface 3142 as shown in FIG. 31D the anterior medial and lateral LPs are angled towards the medial side relative to the bearing axis while the posterior medial and lateral LPs are angled towards the lateral side relative to the bearing axis.

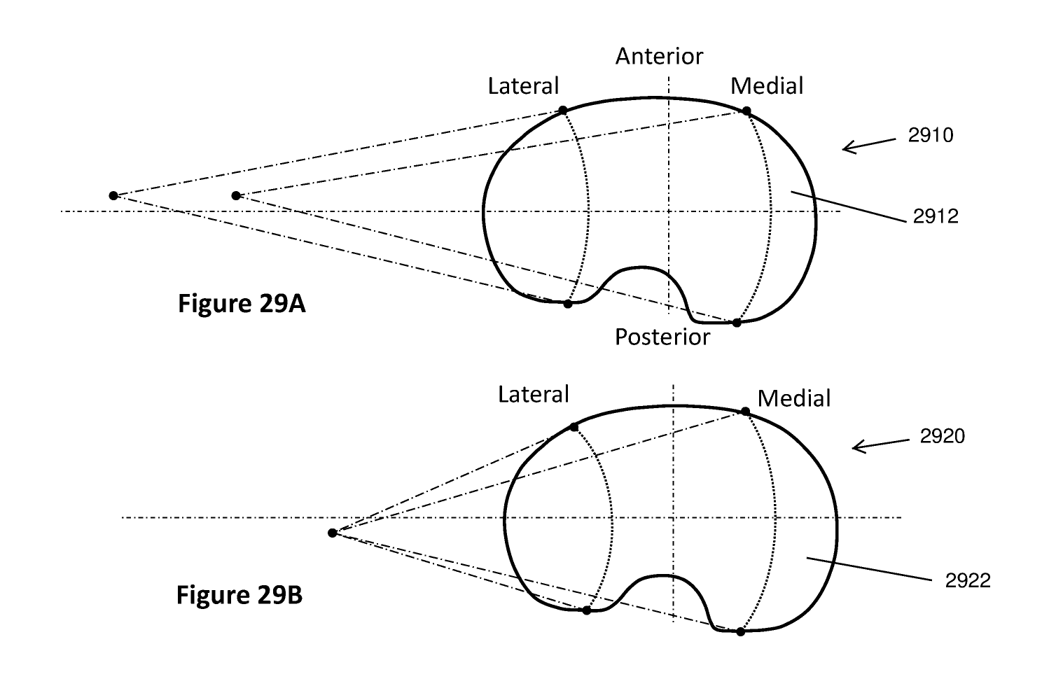

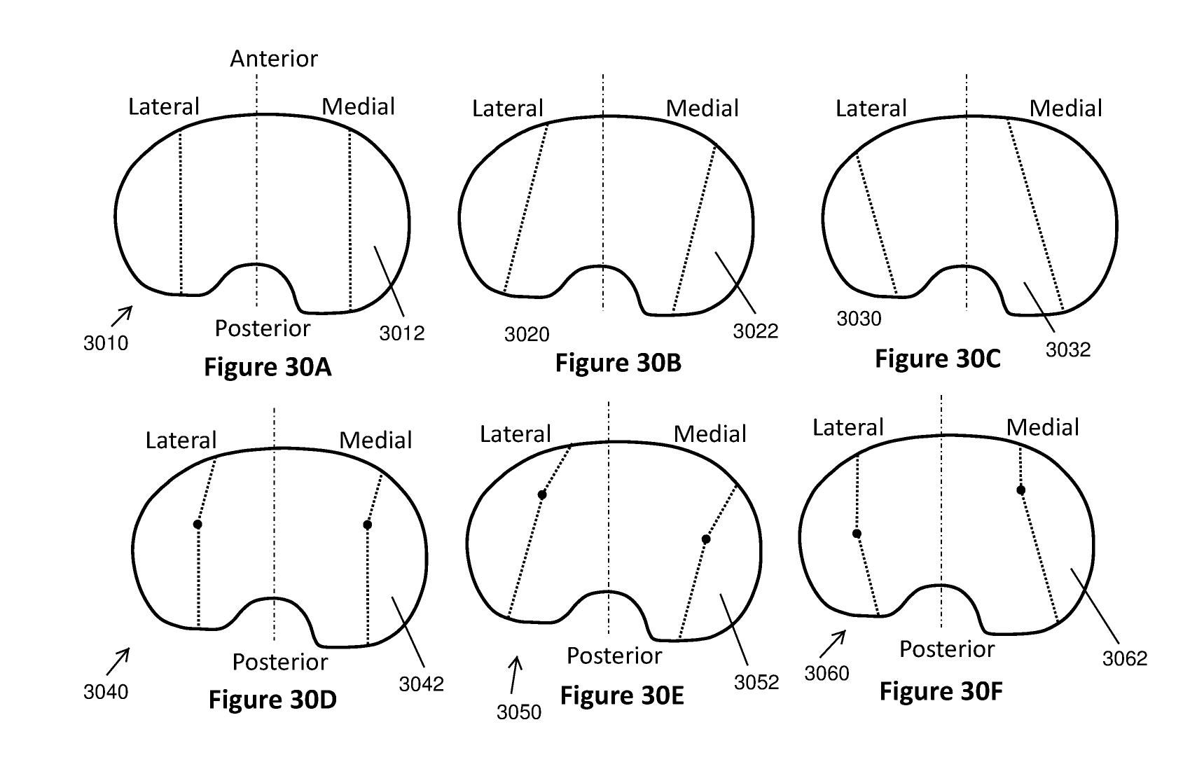

In some embodiments of the invention, the sagittal plane geometry of the medial/lateral tibial bearing articular surface can be varied across the bearings in the library to provide different kinematic patterns. In one embodiment of a tibial bearing 3210 having bearing surface 3212 the medial/lateral bearing articular profile is composed of 2 concave arcs of different radii (FIG. 32A). In another embodiment of a tibial bearing 3220 having bearing surface 3222 the medial/lateral tibial articular profile is composed of an anterior concave arc, a central flat section (line segment) angled relative to the tibial bearing base, and a posterior arc (FIG. 32B). In another embodiment of a tibial bearing 3230 having bearing surface 3232, the medial/lateral tibial articular profile may be composed of an anterior concave arc, and a posterior flat section (line segment) angled relative to the tibial bearing base (FIG. 32C). In another set of embodiments of a tibial bearing 3310 having bearing surface 3312, the medial/lateral tibial articular profile is composed of an anterior concave arc, a central convex arc, and a posterior concave arc (FIG. 33A). In another embodiment of a tibial bearing 3320 having bearing surface 3322, the medial or lateral tibial articular profile is composed of an anterior concave arc, and a posterior concave arc (FIG. 33B). In another embodiment of a tibial bearing 3330 having bearing surface 3332, the medial or lateral tibial articular profile may be composed of a convex arc (FIG. 33C).

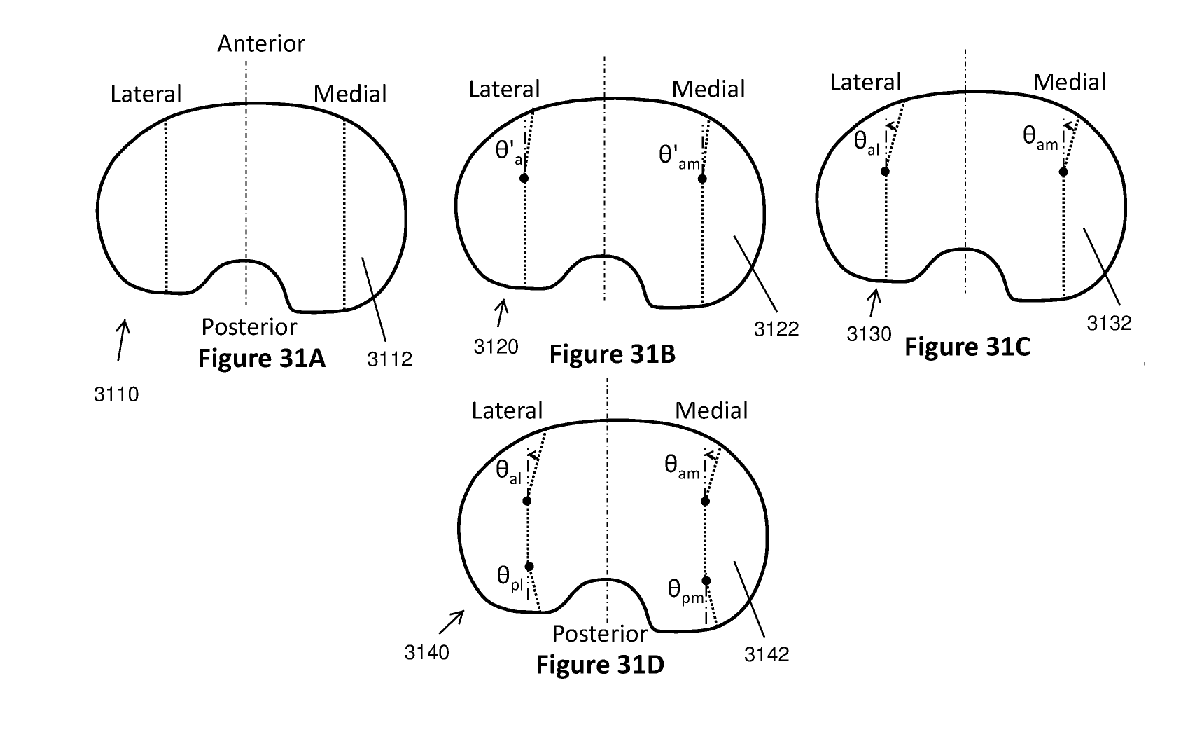

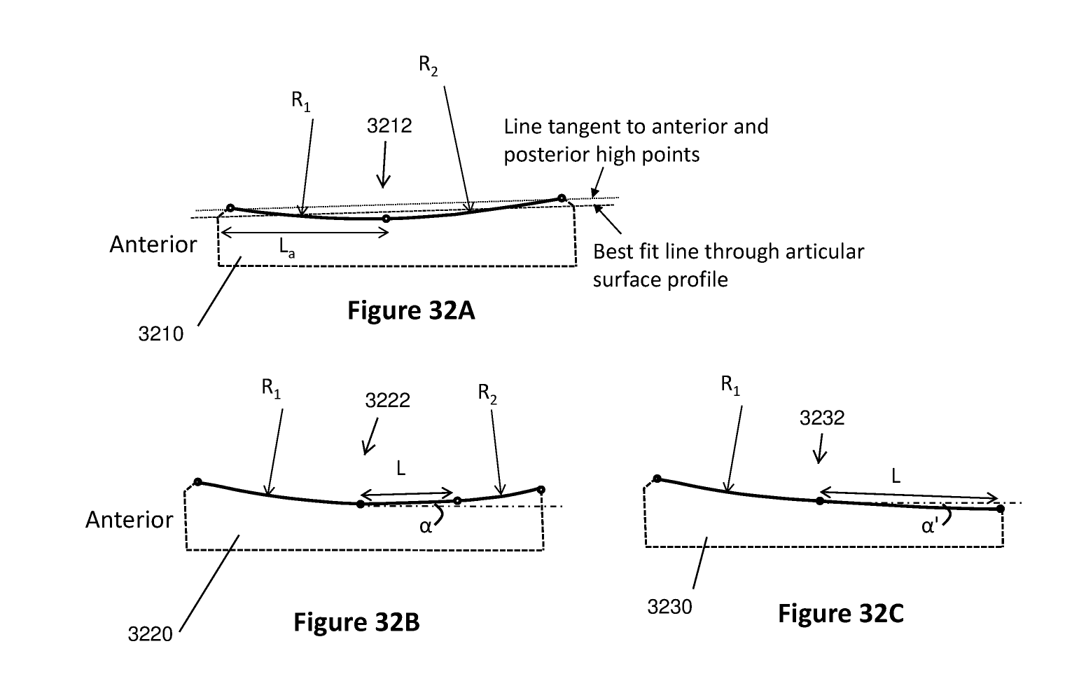

In one set of embodiments of tibial bearings 3410, 3420, 3430 having bearing surfaces 3412, 3422, 3432, bearings of given AP/ML size and PD thickness within the library may be configured to have different medial/lateral articular geometry by changing the slope of a given articular profile relative to the bearing base (FIG. 34A-C). In another set embodiments of a tibial bearing 3510 having bearing surface 3512, bearings of given size and thickness within the library may be configured to have different medial/lateral articular geometry by shifting a given articular profile in anterior or posterior direction(FIG. 35A), shifting the articular low point in anterior or posterior direction of a tibial bearing 3520 having bearing surface 3522, (Lₐ, L′ₐ; FIG. 35B), or shifting the location of intersection of anterior and posterior arcs in anterior or posterior direction of a tibial bearing 3530 having bearing surface 3532 (L_{b}, L′_{b}; FIG. 35C), relative to the tibial bearing base. In another set embodiments, bearings of given size and thickness within the library may be configured to have different medial articular geometry by changing the parameters R₁, R₂, R₃, L, α, α′, Lₐ, L_{b,} L′ₐ, L′_{b,} etc.

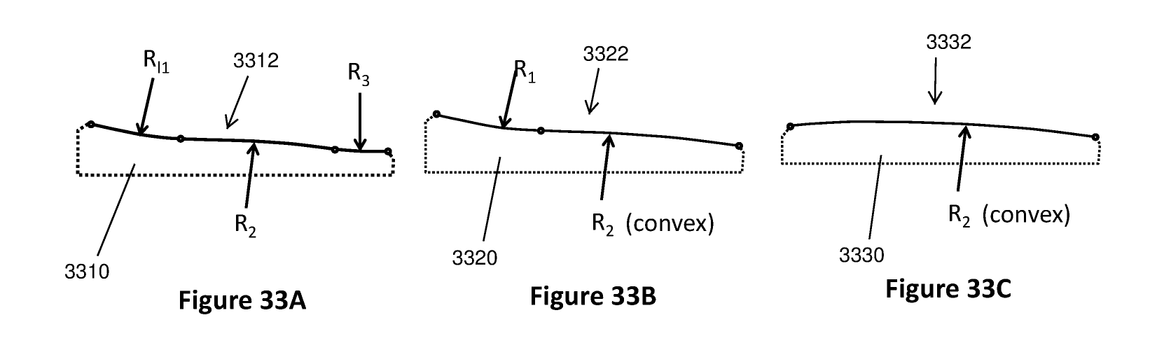

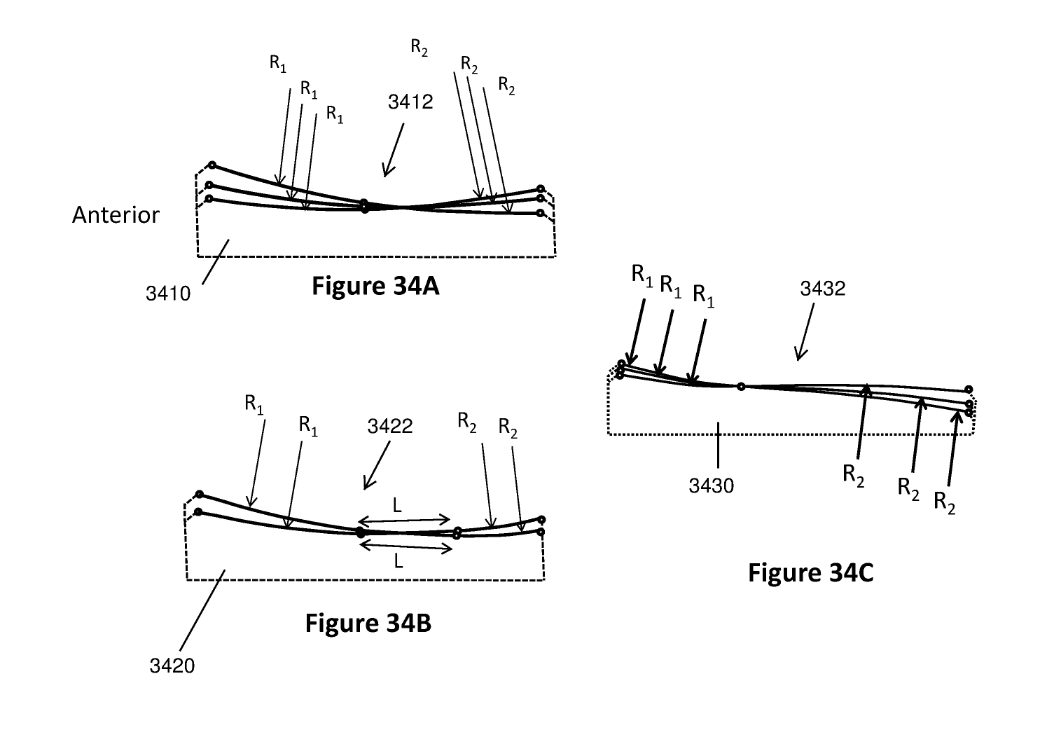

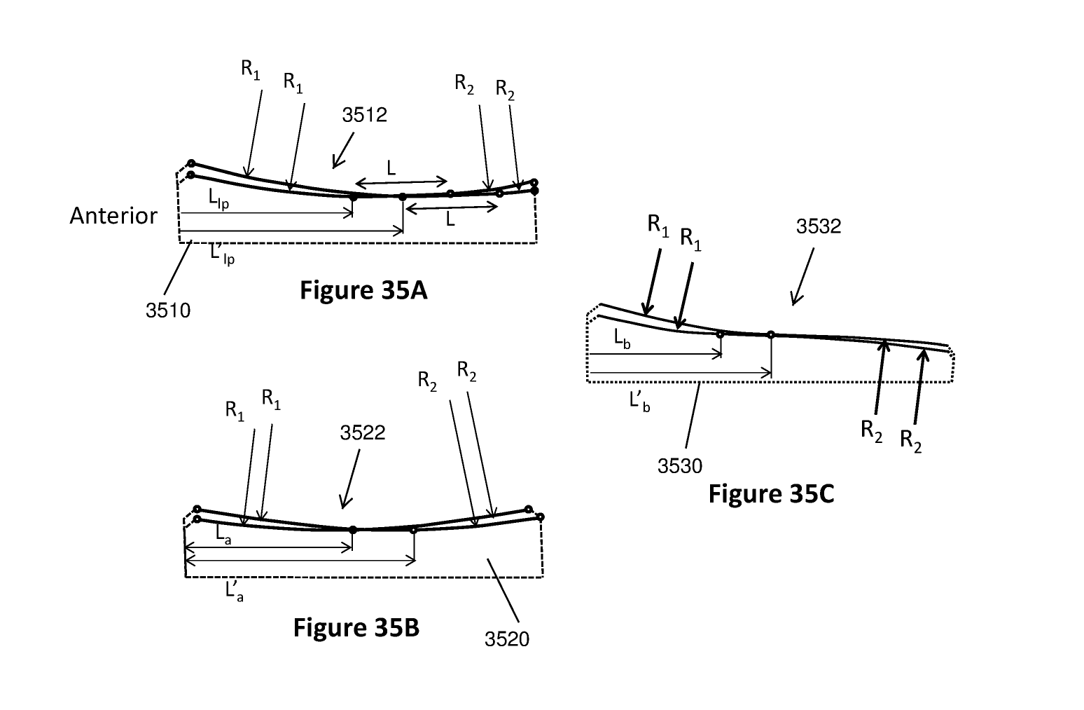

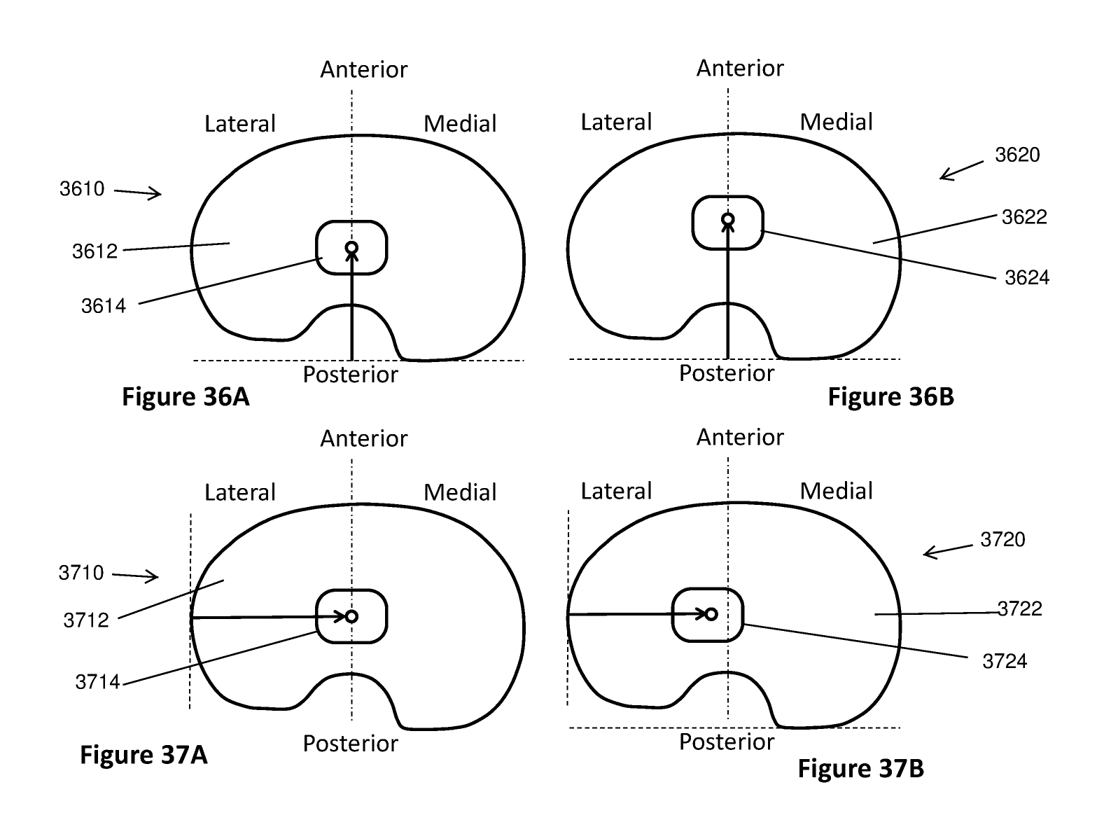

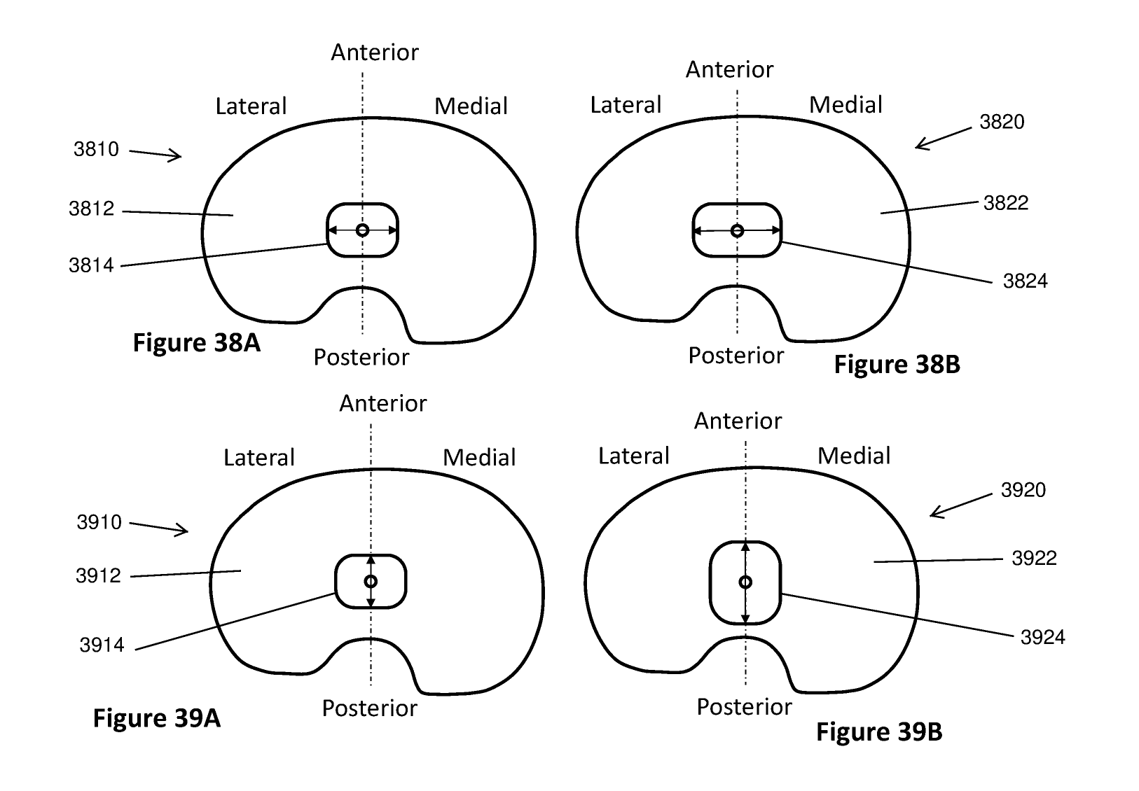

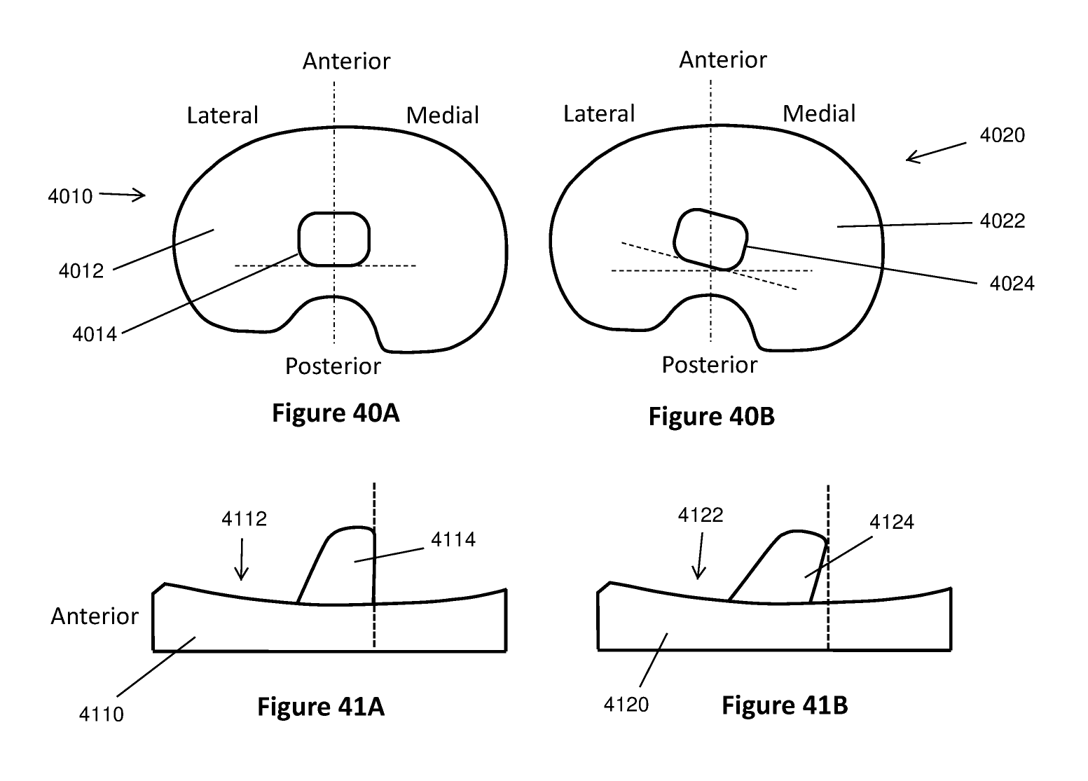

In some embodiments of the invention, the location of an anterior and/or posterior cruciate ligament substituting post can be varied across the bearings in the library to provide different kinematic patterns. In one set of embodiments, bearings 3610, 3620, 3710, 3720 of given size and thickness having bearing surfaces 3612, 3622, 3712, 3722 within the library may be configured to have different anterior/posterior, and/or medial/lateral location of the tibial post (FIG. 36, FIG. 37). In another set embodiments, bearings 3810, 3820, 3910, 3920 of given size and thickness having bearing surfaces 3812, 3822, 3912, 3922 within the library may be configured to have tibial posts 3814, 3824, 3914, 3924 of different anteroposterior width (FIG. 38), different mediolateral width (FIG. 39), and/or different proximal-distal heights, relative to each other. In another set embodiments, bearings 4010, 4020, 4110, 4120 of given size and thickness having bearing surfaces 4012, 4022, 4112, 4122 within the library may be configured to have different orientations of the tibial post 4014, 4024, 4114, 4124 relative to each other, such as different internal and or external rotation in a transverse view (FIG. 40), or different posterior slope in a sagittal view (FIG. 41). In another set embodiments, bearings of given size and thickness within the library may be configured to have different shapes of the anterior, posterior, medial, lateral or proximal surface/s of the tibial post.

In some embodiments, a method of kinematic analysis of acquired image data of a subject for determining geometry of at least one component of a joint prosthesis for the subject is provided. The joint prosthesis can be a total knee replacement. The articulating bone can be a femur. The generic prosthetic component can be a generic femoral component. The articulating surface of the articulating bone can be a distal portion of a femur including a medial condyle and a lateral condyle. The kinematic data of the subject can include range of motion data between maximum flexion of the joint and maximum extension of the joint. The at least one component of the joint prosthesis can be a tibial bearing. The movement of the prosthetic articulating surface through the kinematic data of the subject through a bearing template can carve out the bearing surface of the kinematically appropriate bearing surface from the bearing template through a series of Boolean subtraction operations. An outer profile of the at least one component of the joint prosthesis can be trimmed according to the shape of a baseplate, and locking features are added on the component to mate with the baseplate.

In some embodiments, a fourth database can be created representing stability data of a subject. The fourth database can be merged with the first database representing a two-dimensional or three-dimensional anatomy of an articulating bone of the joint of a subject, the second database representing a two-dimensional or three-dimensional geometry of a generic prosthetic component; the generic prosthetic component being sized to fit around an outer articulating surface of the articulating bone of the joint of the subject; and the third database representing kinematic data of the subject. The prosthetic articulating surface can be stabilized corresponding to the stability data of the subject thereby modifying the bearing template to create a kinematically appropriate and stable bearing surface for the prosthetic articulating surface. The at least one component of a joint prosthesis can be selected based on the modified bearing template having a kinematically appropriate bearing surface from a plurality of joint prosthesis components.

In some embodiments, the kinematically appropriate geometry of at least one component of a joint prosthesis can be determined based on a medial condyle anterior location at a full extension of the joint, a medial condyle posterior location at a maximum flexion of the joint, a lateral condyle anterior location at a full extension of the joint, and a lateral condyle posterior location at a maximum flexion of the joint. The kinematically appropriate geometry of at least one component of a joint prosthesis can be designed with different geometries in the transverse plane, sagittal plane and/or coronal plane.

In some embodiments, the kinematically appropriate geometry of at least one component of a joint prosthesis can have a medial low-point pathway and a lateral low-point pathway. The medial low-point pathway and the lateral low-point pathway can be varied when viewed in a transverse plane.

In some embodiments, the lateral low point-pathway can be parallel to an anterior-posterior axis in a transverse plane, and the medial low-point pathway can be angled in relation to the anterior-posterior axis in the transverse plane.

In some embodiments, an anterior portion of the medial low-point pathway and an anterior portion of the lateral low-point pathway can be angled towards the medial side of the component relative to an anterior-posterior axis and a posterior portion of the medial low-point pathway and a posterior portion of the lateral low-point pathway can be parallel to the anterior-posterior axis in the transverse plane.

In some embodiments, an anterior portion of the lateral low-point pathway can be angled towards the medial side of the knee relative to an anterior-posterior axis, and a posterior portion of medial low-point pathway and a posterior portion of the lateral low-point pathway can be parallel to the anterior-posterior axis in the transverse plane.

In some embodiments, an anterior portion and a posterior portion of the medial low-point pathway and an anterior portion and a posterior portion of the lateral low point pathway can be curved and a central portion of the medial low-point pathway and a central portion of the lateral low-point pathway can be straight and parallel to an anterior-posterior axis in the transverse plane.

In some embodiments, the anterior portion of medial low point pathway and the anterior portion of the lateral low-point pathway can be curved by different amounts with a concave side of the medial low-point pathway and a concave side of the lateral low-point pathway curves facing medially and the posterior portion of the medial low-point pathway and the posterior portion of the lateral low point pathway can be curved by different amounts with a concave side of the medial low-point pathway and a concave side of the lateral low-point pathway curves laterally.

In some embodiments, an anterior portion of the medial low-point pathway and an anterior portion of the lateral low-point pathway can be angled laterally relative to an anterior-posterior axis in the transverse plane and a posterior portion of the medial low-point pathway and a posterior portion of the lateral low-point pathway can be parallel to the anterior-posterior axis in the transverse plane.

In some embodiments, an anterior portion of the medial low-point pathway and an anterior portion of the lateral low-point pathway can be curved in a transverse plane with a concave side of the curve facing laterally, and a posterior portion of the medial low-point pathway and a posterior portion of the lateral low-point pathway can be curved with the concave side of the curve facing medially in the transverse plane.

In some embodiments, the medial low-point pathway and the lateral low-point pathway can be curved in a transverse plane, and a center of curvature medial low-point pathway and a center of curvature of the lateral low-point pathway can be varied.

In some embodiments, the medial low-point pathway and the lateral low-point pathway can be curved with the concave side of the curvature facing medially, and the center of curvature can be different for the medial low-point pathway and the lateral low-point pathway, and each center of curvature can be outside of the component.

In some embodiments, the medial low-point pathway and the lateral low-point pathway can be curved with the concave side of each curve facing medially, the medial low-point pathway and the lateral low-point pathway having a center of curvature that can be the same.

In some embodiments, the medial low-point pathway and the lateral low-point pathway can be curved with the concave side of the curvature facing medially, and the center of curvature of the medial low-point pathway can be located outside of the component and can be different than the center of curvature of the lateral low-point pathway located within the component.

In some embodiments, the medial low-point pathway and the lateral low-point pathway can be curved with the concave side of each curve facing laterally, the medial low-point pathway and the lateral low-point pathway having a center of curvature that can be the same.

In some embodiments, the medial low-point pathway and the lateral low-point pathway can be curved with the concave side of the curvature facing laterally, and the center of curvature can be different for the medial low-point pathway and the lateral low-point pathway, and each center of curvature can be outside of the component.

In some embodiments, the medial low-point pathway can be one or more straight lines oriented at an angle relative to the anterior-posterior axis in the transverse plane, the lateral low-point pathway can be one or more straight lines oriented at an angle relative to the anterior-posterior axis in the transverse plane.

In some embodiments, the bearing surface can have a centrally positioned anterior-posterior bearing axis in the transverse plane, and the medial low-point pathway and the lateral low-point pathway can be angled relative to the bearing axis.

In some embodiments, the bearing surface can have a centrally positioned anterior-posterior bearing axis in the transverse plane, and an anterior portion of the medial low-point pathway and an anterior portion of the lateral low-point pathway can be angled towards the medial side relative to the bearing axis. A posterior portion of the medial low-point pathway and a posterior portion of the lateral low-point pathway can be angled towards the lateral side relative to the bearing axis

In some embodiments, a sagittal plane geometry of the bearing surface can be composed of two concave arcs of different radii. A sagittal plane geometry of the bearing surface can be composed of an anterior concave arc, a central flat section angled relative to a tibial base, and a posterior arc.

In some embodiments, a sagittal plane geometry of the bearing surface can be composed of an anterior concave arc, a central convex arc, and a posterior concave arc.

In some embodiments, a sagittal plane geometry of the bearing surface can be composed of an anterior concave arc, and a posterior concave arc.

In some embodiments, a sagittal plane geometry of a medial portion of the bearing surface can be composed of a convex arc.

In some embodiments, a sagittal plane geometry of a lateral portion of the bearing surface can be composed of a convex arc.

In some embodiments, a sagittal plane geometry of a medial portion of the bearing surface can have a slope that is different than a slope of a sagittal plane geometry of a lateral portion.

In some embodiments, a sagittal plane geometry of a medial portion of the bearing surface can be different than a sagittal plane geometry of a lateral portion by shifting an articular profile in an anterior-posterior direction relative to a tibial base.

In some embodiments, a sagittal plane geometry of a medial portion of the bearing surface can be different than a sagittal plane geometry of a lateral portion by shifting an articular low point in anterior-posterior direction relative to a tibial base.

In some embodiments, a sagittal plane geometry of a medial portion of the bearing surface can be different than a sagittal plane geometry of a lateral portion by shifting a location of an intersection of anterior and posterior arcs in an anterior-posterior direction relative to a tibial base.

In some embodiments, the bearing surface can include a ligament replacement post, the ligament replacement post can be kinematically adjustable in an anterior-posterior and medial-lateral directions in the transverse plane.

In some embodiments, the bearing surface can include a ligament replacement post, the ligament replacement post can be kinematically adjustable in an anterior-posterior width, medial-lateral width, and proximal-distal height in the transverse plane.

In some embodiments, the bearing surface can include a ligament replacement post, the ligament replacement post can be kinematically adjustable rotationally in a transverse plane.

In some embodiments, the bearing surface can include a ligament replacement post, the ligament replacement post can be kinematically adjustable in an anterior-posterior slope in a sagittal plane.

In some embodiments, the joint prosthesis can be a total shoulder replacement. The articulating bone can be a humerus. The generic prosthetic component can be a generic humeral component. The articulating surface of the articulating bone can be a proximal portion of a humerus.

In some embodiments, the joint prosthesis can be a total hip replacement. The articulating surface of the articulating bone can be a proximal portion of a femur.

In some embodiments, the joint prosthesis can be a wrist replacement. In other embodiments, the joint prosthesis can be an elbow replacement. In other embodiments, the joint prosthesis can be an ankle replacement.

In some embodiments, the bearing surface can comprise at least of material selected from the group consisting of polyaryletherketone (PEEK), polyolefins, polyethylene, ultra-high molecular weight polyethylene, medium-density polyethylene, high-density polyethylene, medium-density polyethylene, and highly cross-linked ultra-high molecular weight polyethylene (UHMWPE), and blends thereof.

### Custom (Patient-Specific) Bearings for Other Joints:

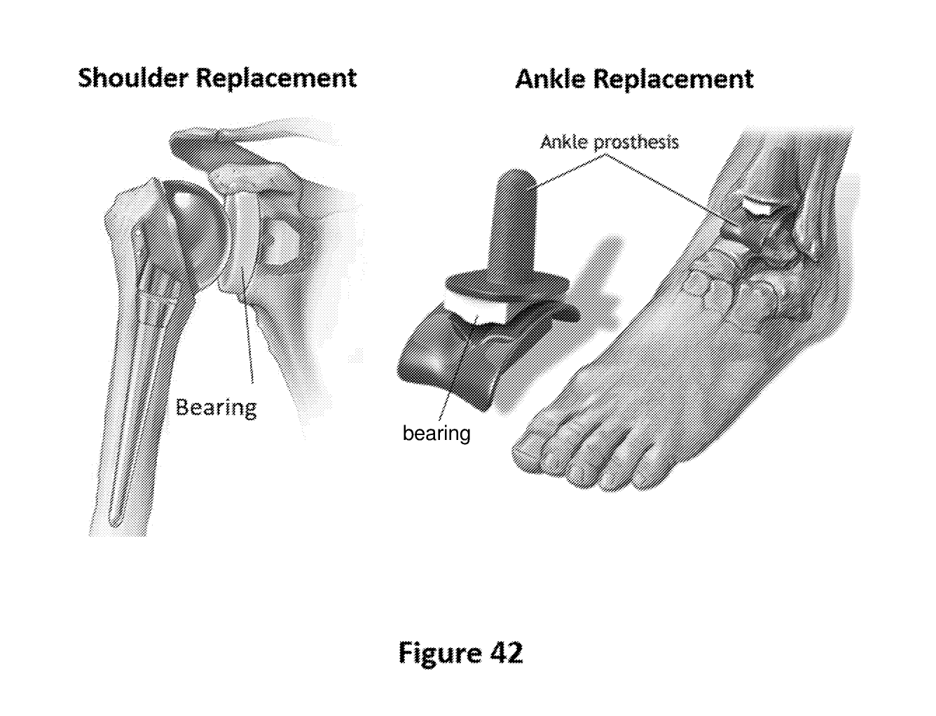

The methods described above may also be used to design patient-specific bearings for other joint replacement implants, including shoulder replacement implants (total shoulder, reverse shoulder etc.), ankle replacement implants, hip replacement implants, wrist replacement implants, elbow replacement implants, etc. (FIG. 42). Such bearings may be coupled with non-patient specific/off-the-shelf components, to create custom-bearing shoulder replacement implants, custom-bearing ankle replacement implants, custom-bearing hip replacement implants, custom-bearing wrist replacement implants, custom-bearing elbow replacement implants etc.

In some embodiments, the joint prosthesis can be a total knee replacement. In some embodiments, the first articulating bone can be a femur. The one or more generic prosthetic components can be a generic femoral component. The first articulating bone can have an articulating surface that can be a distal portion of a femur including a medial condyle and a lateral condyle. The at least one of the one or more patient-specific components can be a tibial bearing.

In some embodiments, the first articulating bone can be a humerus.

In some embodiments, at least one of the one or more patient-specific prosthetic components can comprise a material selected from the group consisting of polyaryletherketone (PEEK), polyolefins, polyethylene, ultra-high molecular weight polyethylene, medium-density polyethylene, high-density polyethylene, medium-density polyethylene, highly cross-linked ultra-high molecular weight polyethylene (UHMWPE), and blends thereof.

### Prosthesis Materials and Construction

The prosthesis components described herein can be constructed in various manners and out of one or more materials. For example, the tibial bearings may be constructed out of materials such as polyaryletherketone (e.g. Polyetheretherketone—PEEK), polyolefins, polyethylene, ultra-high molecular weight polyethylene, medium-density polyethylene, high-density polyethylene, medium-density polyethylene, highly cross-linked ultra-high molecular weight polyethylene (UHMWPE), etc. Exemplary embodiments of UHMWPE prosthesis materials and manufacturing processes are described in U.S. patent application Ser. No. 08/600,744 (now U.S. Pat. No. 5,879,400) filed Feb. 13, 1996, entitled “Melt-Irradiated Ultra High Molecular Weight Polyethylene Prosthetic Devices;” U.S. patent application Ser. No. 12/333,572 filed Dec. 12, 2008, entitled “Radiation And Melt Treated Ultra High Molecular Weight Polyethylene Prosthetic Devices;” U.S. patent application Ser. No. 11/564,594 (now U.S. Pat. No. 7,906,064) filed Nov. 29, 2006, entitled “Methods For Making Oxidation Resistant Polymeric Material;” U.S. patent application Ser. No. 12/522,728 filed Apr. 5, 2010, entitled “Methods For Making Oxidation-Resistant Cross-Linked Polymeric Materials;” U.S. patent application Ser. No. 11/030,115 (now U.S. Pat. No. 7,166,650) filed Jan. 7, 2005, entitled “High Modulus Crosslinked Polyethylene With Reduced Residual Free Radical Concentration Prepared Below The Melt;” U.S. patent application Ser. No. 12/041,249 filed Mar. 3, 2008, entitled “Cross-Linking Of Antioxidant-Containing Polymers;” which are hereby incorporated by reference in their entireties.

The prosthetic components can be machined, cast, forged or otherwise constructed out of a medical grade, physiologically acceptable material such as a cobalt chromium alloy, a titanium alloy, stainless steel, ceramic or the like. Depending on the selection of materials for the different prosthetic components, the designed prosthesis may involve metal on metal articulations, metal on polyethylene articulations, metal on PEEK articulations, ceramic on polyethylene articulations, ceramic on PEEK articulations, ceramic on ceramic articulations, ceramic on metal articulations, polyethylene on polyethylene, PEEK on PEEK articulations etc.

### REFERENCES

1. Kurtz S M, Ong K L, Lau E, Widmer M, Maravic M, Gomez-Barrena E, et al. International survey of primary and revision total knee replacement. Int Orthop. 2011 December; 35(12):1783-9.
2. The orthopedic industry annual report. ORTHOWORLD. 2015.
3. Parvizi J, Nunley R M, Berend K R, Lombardi A V, Jr., Ruh E L, Clohisy J C, et al. High level of residual symptoms in young patients after total knee arthroplasty. Clin Orthop Relat Res. 2014 January; 472(1):133-7.
4. Bourne R B, Chesworth B M, Davis A M, Mahomed N N, Charron K D. Patient satisfaction after total knee arthroplasty: who is satisfied and who is not? Clin Orthop Relat Res. 2010 January; 468(1):57-63.
5. Yue B, Varadarajan K M, Moynihan A L, Liu F, Rubash H E, Li G. Kinematics of medial osteoarthritic knees before and after posterior cruciate ligament retaining total knee arthroplasty. J Orthop Res. 2011 January; 29(1):40-6.
6. Grieco T F, Sharma A, Komistek R D, Cates H E. Single Versus Multiple-Radii Cruciate-Retaining Total Knee Arthroplasty: An In Vivo Mobile Fluoroscopy Study. J Arthroplasty. 2016 March; 31(3):694-701.
7. Johal P, Williams A, Wragg P, Hunt D, Gedroyc W. Tibio-femoral movement in the living knee. A study of weight bearing and non-weight bearing knee kinematics using ‘interventional’ MRI. J Biomech. 2005 February; 38(2):269-76.

The citation of any document is not to be construed as an admission that it is prior art with respect to the present invention.

Thus, the invention provides implants to restore patient-specific function, specifically TKR implants to restore patient-specific knee function.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the embodiments contained herein.

1. A method of manufacturing at least one component of a joint prosthesis, the method comprising:
(a) creating a first database representing a two-dimensional or three-dimensional anatomy of an articulating bone of a joint of a subject;
(b) accessing a second database representing a two-dimensional or three-dimensional geometry of a generic prosthetic component; the generic prosthetic component being sized to fit around an outer articulating surface of the articulating bone of the joint of the subject;
(c) creating a third database representing kinematic data of the subject's joint;
(d) merging the first database, the second database, and the third database within a three-dimensional image based medium wherein the generic prosthetic component is attached to the outer articulating surface of the articulating bone of the joint creating a prosthetic articulating surface, the prosthetic articulating surface being moved through the kinematic data of the subject through a bearing template thereby modifying the bearing template to create a kinematically appropriate bearing surface for the prosthetic articulating surface; and
(e) manufacturing the at least one component of the joint prosthesis to have a geometry corresponding to the modified bearing template having a kinematically appropriate bearing surface.

2. The method of claim 1 wherein:
the joint prosthesis is a total knee replacement.

3. (canceled)

4. (canceled)

5. (canceled)

6. The method of claim 1 wherein:
the kinematic data of the subject includes range of motion data between maximum flexion of the joint and maximum extension of the joint.

7. (canceled)

8. The method of claim 1 wherein:
the movement of the prosthetic articulating surface through the kinematic data of the subject through a bearing template carves out the bearing surface of the kinematically appropriate bearing surface from the bearing template through a series of Boolean subtraction operations.

9. (canceled)

10. The method of claim 1 further comprising:
creating a fourth database representing stability data of a subject;
merging the fourth database with the first database, the second database, and the third database; and
the prosthetic articulating surface being stabilized corresponding to the stability data of the subject thereby modifying the bearing template to create a kinematically appropriate and stable bearing surface for the prosthetic articulating surface.

11. (canceled)

12. (canceled)

13. A method of kinematic analysis of acquired image data of a subject for determining geometry of at least one component of a joint prosthesis for the subject, the method comprising:
(a) creating a first database representing a two-dimensional or three-dimensional anatomy of an articulating bone of the joint of a subject;
(b) accessing a second database representing a two-dimensional or three-dimensional geometry of a generic prosthetic component; the generic prosthetic component being sized to fit around an outer articulating surface of the articulating bone of the joint of the subject;
(c) creating a third database representing kinematic data of the subject;
(d) merging the first database, the second database, and the third database into a three-dimensional image based medium wherein the generic prosthetic component is attached to the outer articulating surface of the articulating bone of the joint of the subject creating a prosthetic articulating surface, the prosthetic articulating surface being moved through the kinematic data of the subject through a bearing template thereby modifying the bearing template to create a kinematically appropriate bearing surface for the prosthetic articulating surface; and
(e) determining a kinematically appropriate geometry of at least one component of a joint prosthesis based on the modified bearing template having a kinematically appropriate bearing surface.

14. The method of claim 13 wherein:
the joint prosthesis is a total knee replacement.

15. (canceled)

16. (canceled)

17. (canceled)

18. The method of claim 13 wherein:
the kinematic data of the subject includes range of motion data between maximum flexion of the joint and maximum extension of the joint.

19. (canceled)

20. The method of claim 13 wherein:
the movement of the prosthetic articulating surface through the kinematic data of the subject through a bearing template carves out the bearing surface of the kinematically appropriate bearing surface from the bearing template through a series of Boolean subtraction operations.

21. (canceled)

22. The method of claim 13 further comprising:
creating a fourth database representing stability data of a subject;
merging the fourth database with the first database, the second database, and the third database; and
the prosthetic articulating surface being stabilized corresponding to the stability data of the subject thereby modifying the bearing template to create a kinematically appropriate and stable bearing surface for the prosthetic articulating surface.

23. The method of claim 13 further comprising:
selecting the at least one component of a joint prosthesis based on the modified bearing template having a kinematically appropriate bearing surface from a plurality of joint prosthesis components.

24. The method of claim 13 wherein:
the kinematically appropriate geometry of at least one component of a joint prosthesis is determined based on a medial condyle anterior location at a full extension of the joint, a medial condyle posterior location at a maximum flexion of the joint, a lateral condyle anterior location at a full extension of the joint, and a lateral condyle posterior location at a maximum flexion of the joint.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The method of claim 13 wherein:
the bearing surface includes a ligament replacement post, the ligament replacement post being kinematically adjustable in an anterior-posterior and medial-lateral directions in the transverse plane.

54. (canceled)

55. (canceled)

56. (canceled)

57. The method of claim 13 wherein:
the joint prosthesis is a total shoulder replacement.

58. (canceled)

59. (canceled)

60. (canceled)

61. (canceled)

62. (canceled)

63. (canceled)

64. (canceled)

65. The method of claim 13 wherein:
the joint prosthesis is an ankle replacement.

66. (canceled)

67. A joint prosthesis comprising:
one or more generic prosthetic components configured to be attached to a first articulating bone of a joint; and
one or more patient-specific prosthetic components configured to be attached to a second articulating bone of the joint,
wherein at least one of the one or more generic prosthetic components articulates against at least one of one or more patient-specific prosthetic components.

68. The joint prosthesis of claim 67 wherein:
the joint prosthesis is a total knee replacement.

69. (canceled)

70. (canceled)

71. (canceled)

72. (canceled)

73. (canceled)

74. (canceled)

75. A method of manufacturing at least one component of a joint prosthesis, the method comprising:
(a) creating a first database representing a two-dimensional or three-dimensional anatomy of an articulating bone of a joint of a subject;
(b) accessing a second database representing a two-dimensional or three-dimensional geometry of a first generic prosthetic component; the first generic prosthetic component being sized to fit around an outer articulating surface of the articulating bone of the joint of the subject;
(c) merging the first database and the second database within a three-dimensional image based medium wherein the first generic prosthetic component is attached to the outer articulating surface of the articulating bone of the joint creating a first prosthetic articulating surface;
(d) creating a third database representing any surface contour deviation of the first prosthetic articulating surface from the outer articulating surface of the articulating bone;
(e) accessing a fourth database representing a two-dimensional or three-dimensional geometry of a second generic prosthetic component for the joint of the subject, the second generic prosthetic component being opposite the first generic prosthetic component;
determining a surface geometry of a second prosthetic articulating surface based on a comparison of the third database and the fourth database; and
(g) manufacturing a bearing surface of a custom prosthetic component to have a geometry corresponding to the second prosthetic articulating surface.

76. The method of claim 75 wherein:
the joint prosthesis is a total knee replacement.

77. (canceled)

78. (canceled)

79. (canceled)

80. (canceled)

81. (canceled)

82. The method of claim 75 wherein:
the surface contour deviation represents one or more of an inward shift and an outward shift of the first prosthetic articulating surface from the outer articulating surface of the articulating bone.
